(19) United States
(12) Patent Application Publication
(10) Pub. No.: US 2003/0232399 A1
(43) Pub. Date: Dec. 18, 2003

(54) Cancer detection methods and reagents

(76) Inventors: John Forsyth Russell Robertson, Attenborough (GB); Catherine Rosamund Louise Graves, Kinoulton (GB); Michael Rawling Price, Nottingham (GB); Frances Margaret Price, Nottingham (GB)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/417,633

(22) Filed: Apr. 16, 2003

Related U.S. Application Data
(63) Continuation-in-part of application No. 09/881,339, filed on Jun. 14, 2001.
(60) Provisional application No. 60/211,886, filed on Jun. 14, 2000.

(51) Int. Cl. G01N 33/574

(52) U.S. Cl. 435/7.23

(57) ABSTRACT

The present invention comprises methods and compositions for detecting cancer in an individual comprising autoantibodies to cancer-associated antigens. Specifically, the present invention comprises methods and compositions for detecting autoantibodies to cancer-associated antigen in a bodily fluid as well as use of said autoantibodies as a means to detect the presence of cancer-associated antigens.

### RELATED APPLICATIONS

This application is a continuation-in-part of U.S. patent application Ser. No. 09/881,339, filed Jun. 14, 2001, which claims the benefit of U.S. Provisional Application Serial No. 60/211,886 filed Jun. 14, 2000, the disclosure of which is incorporated by reference herein in its entirety.

### FIELD OF THE INVENTION

This application relates to immunological reagents and methods for detecting and treating cancer in an animal, most preferably a human. In particular, the invention relates to compositions and methods for detecting or quantitatively measuring the immune response of a mammal to circulating tumour markers or tumour markers expressed on the surface of tumour cells, also to tumour marker antigens for use in these methods and to kits for performing the methods. The present invention also relates to highly sensitive and specific compositions and methods for detecting the presence of cancer or tumour marker proteins in the bodily fluids of a mammal, to autoantibodies for use in these compositions and methods, to immortalised cells for obtaining these autoantibodies and to kits for performing the methods.

### BACKGROUND

The development and progression of cancer in a patient is generally associated with the presence of markers in the bodily fluid of the patient, these “tumour markers” reflecting different aspects of the biology of the cancer (see Fateh-Maghadam, A. & Steilber, P. (1993) Sensible use of tumour markers. Published by Verlag GMBH, ISBN 3-926725-07-9). Tumour markers are often found to be altered forms of the wild type proteins expressed by ‘normal’ cells, in which case the alteration may be a change in primary amino acid sequence, a change in secondary, tertiary or quaternary structure or a change in post-translational modification, for example, abnormal glycosylation. Alternatively, wild type proteins which are up-regulated or over-expressed in tumour cells, possibly as a result of gene amplification or abnormal transcriptional regulation, may also be tumour markers. In some cases, these two phenomena may occur at the same time leading to an accumulation of modified proteins throughout the development of the disease. For example, modified forms of Ras, p53, c-myc, MUC-1 and c-erbB2 have been found to be associated with a wide variety of cancers.

Tumour marker proteins observed to elicit serum autoantibodies include a particular class of mutant p53 protein, described in U.S. Pat. No. 5,652,115, which can be defined by its ability to bind to the 70 kd heat shock protein (hsp70). p53 autoantibodies can be detected in patients with a number of different benign and malignant conditions (described in U.S. Pat. No. 5,652,115) but are in each case present in only a subset of patients. For example, one study utilizing an ELISA assay for detection of autoantibodies directed against the p53 protein in the serum of breast cancer patients reported that p53 autoantibodies were produced by 26% of patients and 1.3% of control subjects (Mudenda, B., Green, J. A., Green, B. et al. The relationship between serum p53 autoantibodies and characteristics of human breast cancer.(1994) Br J Cancer 69: 4445-4449.). A second tumour marker protein known to elicit serum autoantibodies is the epithelial mucin MUC1 (Hinoda, Y. et al. (1993) Immunol Lett. 35: 163-168; Kotera, Y. et al. (1994) Cancer Res. 54: 2856-2860).

In the past the direct detection of cancer-associated proteins has advantageously been used in routine tests for the diagnosis of cancer but, unfortunately, these assays have many limitations. In particular, commercial antibodies available for use in standard tests to detect antigen are usually not sensitive enough to detect the low levels of cancer-associated proteins that are found at the very early stages of the disease, for example in asymptomatic patients, when a treatment would be the most effective. In addition, most commercial antibodies are not specific for modified forms of cancer-associated markers and cross-react with wild-type forms of these proteins. As a consequence, they are only useful for detecting substantial increases in serum levels of cancer marker proteins, which usually occur at advanced stages of cancer.

For example, the commercial assay CA15-3, which detects both unmodified and modified forms of MUC1, is useful in the diagnosis of metastatic breast cancers, which are characterised by elevated serum levels of MUC1. However, this assay cannot be used in screening for neoplasia or primary breast cancer because the serum levels of MUC1 at these stages do not differ significantly from those in normal individuals (Robertson et al. (1990), Eur. J. Cancer 26: 1127-1132). Other marker proteins such as, for example, carcinoembryonic antigen (CEA) and the marker CA19.9 have been reported to be elevated in the serum of patients with metastatic breast and colorectal cancer but not that of patients with primary cancers (Robertson et al. (1991), Cancer Immunol. Immunother. 133: 403-410; Thomas et al. (1991) Br. J. Cancer 63: 975-976). CEA and the glycoprotein termed CA15.3, are also useful as indicators of systemic disease burden and of relapse following therapy (Molina, R., Zanon, G., Filella, X. et al. Use of serial carcinoembryonic antigen and CA 15.3 assays in detecting relapses in breast cancer patients. (1995) Breast Cancer Res Treat 36: 41-48)

Established assays for tumour markers present in bodily fluids tend to focus on the detection of tumour markers which reflect tumour bulk and as such are of value late in the disease process, for example, in the diagnosis of metastatic disease. Additionally, in the case of these cancer markers, available commercial assays are not able to discriminate between modified and wild-type forms of the proteins and are therefore of limited use. Furthermore, commercially available antibodies, by cross-reacting with normal forms of cancer-associated proteins, may also lead to false positive results. Thus, there is a need in the art for more sensitive and specific antibodies to use in these assays in order to detect pre-neoplastic and early carcinogenic modifications.

In most cancers resulting from a progressive accumulation of genetic alterations, such as breast cancer, the presence of tumour markers in bodily fluids reflects the development and progression of disease but no single marker on its own summates all clinically important parameters. For example, the characteristics of a marker useful for diagnosis of cancer may be quite different from markers which convey information about prognosis. Furthermore, in each clinical situation (i.e. diagnosis or prognosis) different markers may be required when dealing with primary cancer and secondary (metastatic) cancer and a different marker again may be required to provide a method of measuring the effectiveness of a particular course of treatment. Different clinical situations therefore require different biological markers and, as has been observed with p53, not all patients express the same set of tumour marker proteins. It is therefore difficult to envisage any one single tumour marker being universally applicable to all patients in all stages of disease. What is needed is the identification of tumour markers or sets of tumour markers present early in the progression of a disease which can be rapidly identified with minimal invasiveness through the use of auto-antibodies.

### SUMMARY OF THE INVENTION

The present invention comprises methods and compositions for identifying and utilizing tumour markers present in bodily fluids that are of use earlier in the disease process and which do not depend on tumour bulk per se.

Differences between a wild type protein expressed by ‘normal’ cells and a corresponding tumour marker protein may, in some instances, lead to the tumour marker protein being recognised by an individual's immune system as ‘non-self’ and thus eliciting an immune response in that individual. This may be a humoral (i.e B cell-mediated) immune response leading to the production of autoantibodies immunologically specific to the tumour marker protein. Autoantibodies are naturally occurring antibodies directed to an antigen which an individual's immune system recognises as foreign even though that antigen actually originated in the individual. They may be present in the circulation as circulating free autoantibodies or in the form of circulating immune complexes consisting of autoantibodies bound to their target tumour marker protein.

As an alternative to the direct measurement or detection of tumour marker protein in bodily fluids, the assays of the present invention measure the immune response of the individual to the presence of tumour marker protein in terms of autoantibody production. Such assays constitute indirect detection of the presence of tumour marker protein. Because of the nature of the immune response, autoantibodies can be elicited by a very small amount of circulating tumour marker protein and indirect methods which rely on detecting the immune response to tumour markers will consequently be more sensitive than methods for the direct measurement of tumour markers in bodily fluids. Assay methods based on the detection of autoantibodies are therefore of particular value early in the disease process and possibly also in relation to screening of asymptomatic patients, for example to identify individuals “at risk” of developing disease.

The present invention comprises methods and compositions comprising a preferred panel of markers involved in the process of carcinogenesis and which have the potential for use in screening and the early diagnosis of primary and recurrent breast cancer initially, but also other cancers. These aberrant proteins are present in small amounts in most patients with early disease but are significantly elevated in only a small minority. Nonetheless, small amounts of such aberrant proteins have the potential to produce an amplified signal through inducing the production of auto-antibodies as a measurable immune response.

Known serum assays, are not optimised for the detection of auto-antibodies, particularly for MUC1. Accordingly, in addition to providing more sensitive assays for cancer diagnosis and prognosis as well as identification of suitable therapies, the present invention comprises a novel means of protein presentation which improves the individual sensitivity of each assay.

It is an object of the present invention to provide an improved assay system for the detection of bodily fluids-borne tumour markers which is more generally useful in all patients and in a variety of different clinical situations.

As used herein the terms “tumour marker protein”, “cancer-associated marker protein”, “marker protein” or “cancer marker” all refer to cancer associated modified forms of wild-type protein and are used interchangeably.

### FIGURES

FIG. 1 provides three charts depicting the results of assays for autoantibodies to p53 (1A), c-erbB2 (1B) and MUC1 (1C), in samples of serum taken from 21 patients diagnosed with primary breast cancer.

FIG. 2 provides three charts depicting: the reactivity profiles of MUC1 protein isolated from normal human urine (2A), ABC MUC1 isolated from the serum of patients with advanced breast cancer (2B) or MUC1 isolated from the human breast cancer cell line ZR75-1 (2C) with various monoclonal anti-MUC1 antibodies.

FIG. 3 provides three graphs depicting the continuous monitoring for recurrent disease in three post-operative breast cancer patients.

FIG. 4 provides two graphs depicting the range of autoantibody levels found in assays for autoantibodies to c-erbB2, c-myc, MUC1 and p53 in normal individuals and patients with early primary breast cancer (PBC).

FIG. 5 provides a chart summarizing the detection rate for primary breast cancer in an analysis of autoantibody levels in a series of healthy controls and patients with primary breast cancer, PBC subdivided by Stage 1—i.e. lymph node negative and Stage 2—i.e. lymph node positive and patients with metastatic cancer at 100% confidence.

FIG. 6 provides a chart summarizing the detection rate for primary breast cancer in an analysis of autoantibody levels in a series of healthy controls and patients with PBC subdivided by Stage 1—i.e. lymph node negative and Stage 2—i.e. lymph node positive and patients with metastatic cancer at 95% confidence.

FIG. 7 provides a chart depicting the sensitivity for primary breast cancer in an analysis of autoantibody levels in a series of healthy controls and patients with Stage 1 or Stage 2 primary breast cancer at 95% confidence.

FIG. 8 provides three graphs depicting the levels of autoantibodies to MUC1, p53 and c-erbB2 in the serum of three patients previously diagnosed with breast cancer measured sequentially during follow-up until the patient manifested recurrent disease.

FIG. 9 provides a graph depicting the autoantibody levels in further samples from the second patient in FIG. 10 (REC at 36 months) taken up to recurrence and during treatment for recurrence.

FIG. 10 provides two graphs depicting follow-up autoantibody levels in post-operative serum samples from two patients, one who did not develop recurrent disease (no REC) and the other who did (REC at 36 months).

FIG. 11 provides a chart summarizing the detection rates in an analysis of autoantibody levels (p53, MUC1, c-erbB2 and c-myc) in samples of serum taken from patients with urologically benign disorders and various stages of bladder cancer.

FIG. 12 provides a chart summarizing the detection rate for colorectal cancer in an analysis of autoantibody levels in the serum of healthy controls, patients with colonic polyps and patients with colorectal cancer at 100% confidence compared to a pre-defined group of healthy controls.

FIG. 13 provides a chart summarizing the detection rate for colorectal cancer in an analysis of autoantibody levels in the serum of healthy controls, patients with colonic polyps and patients with colorectal cancer at 95% confidence compared to a pre-defined group of healthy controls.

FIG. 14 provides a chart summarizing the detection rate in an analysis of autoantibody levels in the serum of healthy controls, patients with primary breast cancer and asymptomatic women known to be BRCA1 mutant carriers at 100% confidence compared to a pre-defined group of healthy controls.

FIG. 15 provides a chart summarizing the detection rate for prostate cancer in an analysis of autoantibody levels in the serum of healthy controls and patients with prostate cancer at 95% confidence compared to a pre-defined group of healthy controls.

FIG. 16 provides a chart summarizing the results of an ELISA assay to examine the reactivity of autoantibodies produced by B cells derived from six patients diagnosed with breast cancer (1 to 4, with primary breast cancer, 7 and 11 with advanced breast cancer).

FIG. 17 provides a chart summarizing the results of an ELISA assay to assess the reactivity of autoantibodies obtained from B cells derived from patients diagnosed with primary breast cancer with MUC1 protein from different sources.

FIG. 18 provides a graph depicting the results of a surface plasmon resonance experiment to measure the binding of autoantibodies produced by B cells derived from patients diagnosed with primary breast cancer to MUC1 protein isolated from the serum of patients with advanced breast cancer (18a) or from the urine of normal individuals (18b).

FIG. 19 depicts the sequence of the peptide that was used to immunoaffinity-purify MUC1 antibodies from the sera of patients with advanced breast cancer.

FIG. 20 provides a chart depicting the results of an ELISA assay employing immobilised autoantibodies from (1) a comparative example of utilising the anti-MUC1 C595 antibody; a patient with (2) primary breast cancer or (3) advanced breast cancer to detect MUC1 protein purified from the serum of a patient diagnosed with advanced breast cancer or from the urine of a healthy individual.

FIG. 21 provides a chart of the results of an ELISA assay utilising immobilised autoantibodies from the B cells of patients with primary breast cancer to detect MUC1 protein in serum samples from healthy individuals or from patients diagnosed with primary or advanced breast cancer.

FIG. 22 provides a chart of the results of an ELISA assay using immobilised autoantibodies from the B cells of patients with primary breast cancer to detect MUC1 protein in sequential serum samples from a patient with advanced breast cancer throughout the progression of the disease.

FIG. 23 provides two charts depicting the results of a number of determinations of the reactivity of sera from breast cancer patients with ABC MUC1 and urinary MUC1.

FIG. 24 provides a chart depicting the ABC serum IgG response to urinary and ABC MUC1.

FIG. 25 provides a chart depicting the ABC serum IgM response to urinary and ABC MUC1.

FIG. 26 provides a chart depicting the results of a sandwich ELISA using human auto-antibodies as a reagent MUC1 (normal and tumour-associated).

### DETAILED DESCRIPTION OF THE INVENTION

The present invention comprises compositions and methods are useful in the early detection of carcinogenic or pre-neoplastic modifications in asymptomatic patients, in monitoring the progress of cancer, in screening for recurrence of the disease in patients who have previously undergone anti-cancer treatment, in monitoring the efficacy of a systematic treatment in a patient and in determining the most appropriate treatment for a particular patient.

The present invention comprises methods and compositions for detecting the immune response of a mammal to circulating tumour marker proteins or tumour cells expressing said tumour marker proteins, comprising:

(a) contacting a sample of bodily fluids from said mammal with a panel of two or more distinct tumour marker antigens;

(b) determining the presence or absence of complexes of said tumour marker antigens bound to autoantibodies present in said sample of bodily fluids, said autoantibodies being immunologically specific to said tumour marker proteins,

whereby the presence of said complexes is indicative of the immune response to circulating tumour marker proteins or tumour cells expressing said tumour marker proteins.

A method of the invention, which may be hereinafter referred to as a ‘panel assay’, utilises a panel of two or more tumour marker antigens to monitor the overall immune response of an individual to a tumour or other carcinogenic/neoplastic changes. In the panel assay, the measurement of any complexes of autoantibodies may be carried out simultaneously for the two or more tumour marker proteins or the presence of autoantibodies against each marker may be measured in separate experiments and the results interpreted together after. This method thus provides essentially a ‘profile’ of the immune response for that individual, indicating which tumour markers elicit an immune response resulting in autoantibody production. The methods of the present invention may be used for the detection of an immune response resulting in the production of circulating free autoantibodies.

Because the assay methods of the invention are performed on a sample of bodily fluids taken from the patient they are essentially non-invasive and can be repeated as often as is thought necessary to build up a profile of the patient's immune response throughout the course of disease or in the case of individuals with a family history, throughout the monitoring period prior to developing the disease. As used herein the term ‘bodily fluids’ includes plasma, serum, whole blood, urine, sweat, lymph, faeces, cerebrospinal fluid, nipple aspirate or other fluids associated with cancer sites. The type of bodily fluid used may vary depending upon the type of cancer involved and the use that the assay is being put to. In general, it is preferred to perform the method on samples of serum or plasma.

As will be illustrated in the Examples given below, the use of a panel of two or more tumour marker antigens to monitor autoantibody production is more sensitive than the use of single markers and gives a much lower frequency of false negative results.

The actual steps of detecting autoantibodies in a sample of bodily fluids may be performed in accordance with immunological assay techniques known per se in the art. Examples of suitable techniques include ELISA, radioimmunoassays and the like. In general terms, such assays use an antigen which may be immobilised on a solid support. A sample to be tested is brought into contact with the antigen and if autoantibodies specific to the tumour marker protein are present in the sample they will immunologically react with the antigen to form autoantibody-antigen complexes which may then be detected or quantitatively measured. Detection of autoantibody-antigen complexes is preferably carried out using a secondary anti-human immunoglobulin antibody, typically anti-IgG or anti-IgM, which recognise general features common to all human IgGs or IgMs, respectively. The secondary antibody is usually conjugated to an enzyme such as, for example, horseradish peroxidase (HRP) so that detection of autoantibody/antigen/secondary antibody complexes is achieved by the addition of an enzyme substrate and subsequent colorimetric, chemiluminescent or fluorescent detection of the enzymatic reaction products.

A panel assay of the present invention uses a panel of tumour marker-related antigens. The panel may be tailored to detect a particular cancer, or a cancer at a particular stage of development. The tumour marker antigens may be wild type or mutant tumour marker proteins isolated from samples of biological fluid from normal individuals or from cancer patients or from cell lines expressing the tumour marker protein, or they may be full length recombinant tumour marker proteins, viral oncogenic forms of tumour marker proteins or antigenic fragments of any of the aforementioned proteins. The term ‘antigenic fragment’ as used herein means a fragment which is capable of eliciting an immune response.

The panel assays may be performed in a multi-well format in which each one of the two or more antigens is placed in separate wells of multi-well assay plates or, alternatively, in a single-pot format in which the entire panel of antigens is placed in a single container. The panel assays may be performed in a qualitative format in which the objective is simply detection of the presence or absence of autoantibodies or in a quantitative format which provides a quantitative measurement of the amount of autoantibodies present in a sample.

Preferred markers for inclusion into the panel of tumour marker antigens include the epidermal growth factor receptor-related protein c-erbB2 (Dsouza, B. et al. (1993) Oncogene. 8: 1797-1806), the glycoprotein MUC1 (Batra, S. K. et al. (1992) Int. J. Pancreatology. 12: 271-283) and the signal transduction/cell cycle regulatory proteins Myc (Blackwood, E. M. et al. (1994) Molecular Biology of the Cell 5: 597-609), p53 (Matlashewski, G. et al. (1984) EMBO J. 3: 3257-3262; Wolf, D. et al. (1985) Mol. Cell. Biol. 5: 1887-1893) and ras (or Ras) (Capella, G. et al. (1991) Environ Health Perspectives. 93: 125-131), including the viral oncogenic forms of ras which can be used as antigens to detect anti-ras autoantibodies, and also BRCA1 (Scully, R. et al. (1997) PNAS 94: 5605-10), BRCA2 (Sharan, S. K. et al. (1997) Nature. 386: 804-810), APC (Su, L. K. et al. (1993) Cancer Res. 53: 2728-2731; Munemitsu, S. et al. (1995) PNAS 92: 3046-50), CA125 (Nouwen, E. J. et al. (1990) Differentiation. 45: 192-8) and PSA (Rosenberg, R. S. et al. (1998) Biochem Biophys Res Commun. 248: 935-939). Additional markers which might also be used include CEA gene family members, PTH-RP, CYFRA21-1, kallikrein, pro-gastrin, gastrin G17, gastrin G34, CA19-9, CA72-4, vasopressin, gastrin releasing peptide, SCC, TK, αFP, p62, annexins I and II, Hv and KOC or antigens of HPV, preferably sub-types associated with cancer risk. As aforementioned, the assays can be performed using tumour marker antigens which are forms of these proteins isolated from human bodily fluids or from cultured cells or antigenic fragments thereof or full length or truncated recombinant proteins or antigenic fragments thereof.

Preferably the tumour marker antigens are labelled with biotin so that they can easily be attached to a solid support, such as a multi-well assay plate, by means of the biotin/avidin or biotin/streptavidin or biotin streptavidin/avidin derivative interaction. Tumour marker antigens labelled with biotin may be referred to herein as ‘biotinylated’ proteins. To facilitate the production of biotinylated tumour marker antigens for use in the assay methods of the invention, cDNAs encoding a full length recombinant tumour marker protein, a truncated version thereof or an antigenic fragment thereof may be expressed as a fusion protein labelled with a protein or polypeptide tag to which the biotin co-factor may be attached via an enzymatic reaction. A useful system for the expression of biotinylated fusion proteins is for instance the PinPoint™ system supplied by Promega Corporation, Madison Wis., USA. Biotinylated tumour marker antigens are able to detect autoantibodies in a much higher percentage of patients than is observed using non-biotinylated antigen.

The assay methods of the present invention may be employed in a variety of different clinical situations such as, for example, in the detection of primary or secondary (metastatic) cancer, in screening for early neoplastic or early carcinogenic change in asymptomatic patients or identification of individuals ‘at risk’ of developing cancer (particularly breast cancer, bladder cancer, colorectal cancer or prostate cancer) in a population of asymptomatic individuals, in the detection of recurrent disease in a patient previously diagnosed as carrying tumour cells who has undergone treatment to reduce the number of tumour cells, in predicting the response of an individual with cancer to a course of anti-cancer treatment or in selection to the said treatment.

The assay methods of the present invention are suitable for detection of many different types of cancer, including, but not limited to, breast, bladder, colorectal, prostate, pancreatic, ovarian, liver, lung, gastric, endometrial and cervical as well as cancers of the skin. The assays of the present invention may complement existing methods of screening and surveillance. For example in the case of primary breast cancer it could be used to alert clinicians to biopsy small lesions on mammograms which radiographically do not appear suspicious or to carry out breast imaging or to repeat imaging earlier than planned. In the clinic, the assay methods of the present invention are more objective and reproducible compared to current imaging techniques (i.e. mammography and ultrasound), the success of which can be operator-dependent.

Breast Cancer

Breast cancer is the most common malignancy affecting women and is a major cause of death in women in Western Europe and the USA. Incidence rates are highest in the developed nations, where one in eight women, and one in ten women, develop breast cancer in the USA and Western Europe respectively. Nor is this rate static, indeed it has risen steadily over the past 50 years and continues to increase at approximately 1% annually. Due to improvements in detection and awareness, only a minority of women (10%) now present with distant metastasis, yet up to 50% of patients still progress to advanced disease from which the majority will ultimately die. Predictions for the world-wide incidence of breast cancer over the next decade indicate that 5 million people will be affected. The development and progression of breast cancer in patients is known to be associated with the presence of markers in their blood. Each of these blood “tumour markers” reflect different aspects of the biology of breast cancer and no single tumour marker summates all clinically important parameters. For example the characteristics required of a blood marker for diagnosis are quite different from those which convey information about prognosis. Furthermore, in each situation (i.e. diagnosis and prognosis) different markers may be required when dealing with primary and secondary (metastatic) breast cancer. Similarly different blood markers again may be required to provide a method of measuring the effectiveness of treatment. Different clinical situations therefore require different biological markers.

In each area where established blood markers are currently useful in breast cancer, combinations of markers are required rather than a single marker, reflecting the complexity and heterogeneity of breast cancers. It is most likely that in new clinical areas where blood markers are being investigated, combinations of markers may again prove better than any single marker.

Established Markers

To date established blood tumour markers reflect tumour burden in that they are useful:

for diagnosis of metastatic disease;

for monitoring whether metastases are responding or progressing on treatment;

for earlier detection of recurrence.

However no blood markers have been established to be of value earlier in the course of the disease, i.e. for screening and early diagnosis of primary breast cancer. Evidence can be cited in support of the view that earlier detection, both of primary and metastatic disease, is clinically worthwhile. Mammographic screening has been shown to reduce mortality and morbidity (e.g. smaller tumours allow breast conserving surgery rather than radical surgery) from breast cancer. It is also established that adjuvant therapy prolongs survival compared to waiting and using the same therapy when symptomatic metastasis are diagnosed ^{1,2}. Adjuvant therapy can be used in the treatment of patients post-surgery who appear disease-free clinically but in whom there is other evidence of metastasis (i.e. elevation of serum markers). This applies irrespective or not of whether patients received standard adjuvant therapy. Small pilot studies of such a hypothesis have been reported or are currently being carried out^{3,4,5,6}.

The use of serum markers for measuring tumour burden and monitoring therapy is supported by a number of studies in advanced disease⁷⁻¹³. The present invention comprises an investigation of the role of blood tumour markers in the detection of advanced breast cancer and then using changes in the markers to monitor systemic therapy in patients with metastases.

The development of molecular biology has increased understanding of early tumourgenesis and thereby identified potential new markers which might be useful for screening and early diagnosis. These markers are based on the understanding of genetic abnormalities associated with breast cancers, the role of growth factors, cellular transcription factors and the cell cycle. Initially, 4 markers were selected associated with early carcinogenesis; MUC1, p53, c-erbB2 and c-myc. For each marker the following studies were performed:

serum measurements of i) antigen and ii) auto-antibody;

tumour measurements of antigen expression (±mutations).

Abnormally glycosylated MUC1 mucins are already of clinical value in advanced breast cancer but they are as yet of no value in the primary disease due to the insensitivity of the current assays. However, it has recently been reported that 10-25% of patients with primary tumours express antibodies to MUC1 mucins^{14,15}. These antibodies were found in patients blood either as free antibody, or complexed to the mucin molecule. Those patients positive for circulating immune complexes have a significantly greater disease free survival period than those deemed negative. Also reported is the identification of MUC1 in immune complexes isolated from the sera of breast cancer patients. It is therefore clear that an antibody response occurs before the level of the mucin itself is elevated in the serum thus making anti-MUC1 auto-antibodies useful diagnostic blood marker in early disease.

Up to 50% of pre-treatment primary breast cancer patients have elevated antibodies in their blood reactive with the MUC1 mucin antigen. To achieve this increased level of sensitivity and rate of detection, it is preferable to use a MUC1 antigen preparation purified from the serum of patients with advanced breast cancer, and this antigen is clearly a more appropriate target for screening patient serum that the synthetic and ‘normal’ MUC1 mucin material used previously. Assays for the detection of auto-antibodies to p53, c-erbB2 and c-myc, and more recently to BRCA1 and PSA, have been designed utilising recombinant proteins which have been produced with an N terminal biotin tag. By using avidin coated plates it is then possible to attach the ‘antigen’ to the plate in such a way that the majority of the protein is available in a non-constrained conformation. This has led to the increased sensitivity of each individual assay. Standard operating procedures are already in place for sample collection, aliquoting and storage, as well as for the assay procedures.

There are significant differences between the MUC1 molecules derived from normal individuals and those derived from cancer patients, which appear to depend on the stage of the cancer, and these are currently utilised in order to enhance the sensitivity of MUC1 assays. These differences include both the confirmation of the MUC1 molecule and its immunogenicity with respect to the human immune system. As a further part of this work human monoclonal anti-tumour marker antibodies have been developed (see below) for use in diagnostic assays for early breast cancer. Epitope mapping on 56 mouse monoclonal anti-MUC1 antibodies and 24 human anti-MUC1 auto-antibodies has been performed. Those regions of the MUC1 molecule that are immunodominant in the mouse are not the most immunodominant regions in the human situation. Developed human monoclonal antibodies are more specific to human tumour associated proteins than are the currently utilised mouse monoclonals.

As will be discussed below, the panel of markers may include BRCA1, BRCA2 and PSA. BRCA1 and 2 provide specificity for breast (and ovarian) cancer. PSA (normally elevated in prostate cancer in men) is also known to be associated with some breast cancers and may therefore be tissue specific in women.

The assays of the invention are useful in the diagnosis of primary breast cancer, e.g. assessing ‘low risk’ mammographic abnormalities identified through mammographic screening. Initial clinical applicability of these is in two areas although others are not excluded.

‘At Risk’ Population

There is an increasing awareness that some women are at higher risk of developing breast cancer than others. This has resulted in a large increase of women presenting themselves for screening. None of the current options are ideal. Mammographic screening detects breast cancer once it has developed, and in some identifiable groups of women (e.g. young women) in 40-50% of patients after it has metastasised to regional lymph nodes. Gene testing for high penetrance breast cancer susceptibility genes BRCA1 and 2 are informative in less than 5% of patients. Even where women are identified as BRCA gene mutation carriers only 70% will ever develop a breast cancer in their lifetime. Furthermore, only 50% will develop breast cancer before 50 years of age. There is therefore a need both for preventative strategies per se but also for strategies which take account of the timing of intervention.

As mentioned earlier, patients with a confirmed family history of breast cancer are currently treated in one of three ways:

1. Increased surveillance (i.e. usually by regular mammography);

2. Drug ‘prevention’ (e.g. Tamoxifen); or

3. Surgical removal of organs at risk (e.g. bilateral mastectomy+/−bilateral oophorectomy).

Currently, the decision to treat a patient with a family history of breast cancer is based on the basis of probability. Nevertheless, most patients choose one of the above three options, including radical surgery. It is of note that BRCA1 and BRCA2 mutations account for only 5% of breast cancer. There is still a further 15% of patents who have a strong family history in whom no known gene mutation will be identifiable. These patients are also prepared to decide on options 1-3 above based purely on the family history, with no further biological information. The use of a panel of markers in such situations provides stronger biological and statistical evidence of cancer risk and/or of the early diagnosis of cancer than is currently available to such women. Elevation either of site specific auto-antibodies or of auto-antibodies such as p53 and c-erbB2, would provide evidence for more intensive/frequent imaging with greater sensitivity than mammography (e.g. MRI, PET) and/or therapeutic intervention. Given that at present most patients with a family history make decisions based solely on a theoretical risk calculation, auto-antibodies will provide much needed biological evidence.

Preventative strategies (e.g. radical surgery or chemoprevention) still remain unproven while at the same time either involve mutilating surgery or drugs with significant side-effects. There is an urgent need for effective strategies in this population of women. The present invention comprises assays for the detection of auto-antibodies to a truncated BRCA1 protein which has achieved an anti-BRCA1 auto-antibody detection rate of 54% in BRCA1 mutation positive serum samples, in pilot studies. When combined with assays for the detection of auto-antibodies to MUC1, p53,c-erbB2 and c-myc the detection rate increases to 82% (72% without BRCA1), again in pilot studies. Advantageously, truncated forms of BRCA1 and BRCA2 proteins can be included as antigens in auto-antibody assays. Sequential measurements with these assays should both identify the women in whom prevention should be initiated and provide guidance as to the treatment.

Following the development of MUC1, p53, c-erbB2 and c-myc auto-antibody assays, a well characterised series of patient sera has been analysed, 100 normal healthy individuals, 200 patients with primary breast cancer (serum and tumour samples), 50 patients with advanced breast cancer and a number of sequential samples from patients with progressive and non-progressive disease.

Data analysis has revealed that by combining the results of each assay and allocating patients a score between 0/4 (negative in all 4 assays) and 4/4 (positive in all 4 assays) a sensitivity for primary breast cancer detection of 75-85% positive in at least one out of four assays, with a specificity of 100%, has been achieved. In other words, a simple test on a blood sample taken shortly after normal clinical diagnosis of breast cancer was able to detect cancer in 75-85% of the patients analysed. This far exceeds any results previously achieved using conventional antigen based assays.

BRCA1 Mutation Carriers

Serum samples from a small sub-set of patients known to carry BRCA1 mutations, were analysed. Using a five assay panel (BRCA1, MUC1, p53, C-erbB2 and c-myc) 82% of these individuals were positive in one or more of the assays.

Development of such a panel of markers are used for a number of clinical situations. They are described in relation to breast cancer, but they are applicable to other tumour types too. The applications include, but are not limited to:

SCREENING AND EARLY DIAGNOSIS—for the foreseeable future the final diagnosis of primary breast cancer will continue to be histological. However, blood tumour markers which accurately detect early disease could be useful as a screening method to identify which women should be further investigated. This would be particularly valuable in younger women where the value of mammographic screening is not established. In older women a blood test could be used to compliment mammography or alternatively as an initial screening modality. As regard the former one, potential application of these assays would be to alert patients and clinicians to biopsy small lesions detected for example on mammographic screening programmes.

As regards the latter, a panel of auto-antibody assays can detect very early carcinogenesis whereas mammography detects established larger tumours. A panel of autoantibodies could therefore be used as an initial screen (alone or in combination with a screening modality such as mammography). Where autoantibodies were detected and the cancer not identified by mammography, more sensitive imaging modalities (e.g. MRI, PET) could be carried out to locate the tumour. Where even these modalities were negative the following alternative strategies could be adopted:

i) Sequential frequent screening with the most sensitive imaging tests available (e.g. MRI, PET);

ii) Chemoprevention;

iii) Radical surgery

RISK ASSESSMENT—current clinical; strategies for patients at risk of breast cancer (e.g. strong Family History) involve i) regular follow up and mammography; (ii) chemoprevention (e.g. tamoxifen) or iii) prophylactic surgery (i.e. bilateral mastectomy±bilateral oophorectomy). All of these approaches have significant disadvantages. The strategy of regular follow-up and mammography involves the detection of cancers once they have developed. Current treatment strategies aimed at prevention in these patients are based on risk assessment since most of the patients being followed up will never develop breast cancer. Therefore, whilst a patient may be at ‘statistical risk’ because of family history, few are at ‘biological risk’. The assays of the present invention will provide better biological information on which to base clinical advice and decisions. The strategy of prophylactic surgery is mutilating for women who at this time point are well and healthy, affecting their femininity and self esteem, and yet still does not provide a 100% cure. A sensitive and specific screening method would allow patients and clinicians to make decisions based on biological risk assessment of what was currently happening in respect to individual patients. In addition to using antibodies as a biological risk measurement, a strategy similar to that described above (SCREENING AND EARLY DIAGNOSIS—final paragraph in this section) could be introduced using mammography, MRI and/or PET with options i)-iii) for those women who have autoantibodies but are breast imaging negative.

POST-SURGERY FOLLOW-UP—current markers (e.g. CA15-3 and B27.29) reflect tumour bulk and are a manifestation of occult metastasis. Both CA15-3 and CA27.29 have a median lead time of approximately 6 months. The auto-antibody assays of the invention have the capacity to detect auto-antibodies elevated much earlier through a secondary immune response to antigen, present in a small volume of recurrent disease, against which the patient had already made an immune response to during the primary stages of disease.

The above clinical applications are in the area of screening and early diagnosis where current serum markers have little to offer. It is believed that no single tumour marker will be sufficient due in the main to the heterogeneity of breast and other solid cancers. The present invention comprises compositions and methods for new marker assays and takes into consideration the fact that a panel of markers is more likely to be successful than any single marker.

Other Uses

There are other potential uses for these markers in breast cancer which would include (but are not limited to):

selecting for therapy;

predicting and measuring response to treatment;

defining individualised vaccination protocols;

monitoring vaccination programmes;

host specific radioimmunoscintigraphy;

host specific immuno-targeted therapy.

The description and further characterisation of these markers also provides a means of developing human antibodies for immunotargeting, both for diagnostic and therapeutic purposes as well as valuable information for the development of cancer vaccines.

Benefits

Health care systems stands to accrue substantial benefit in a number of areas if the current methods of screening and surveillance for development of breast cancer are superseded by serum tumour markers.

Weaknesses of current methods include:

Current care is based on risk assessment. The strongest of these (i.e. family history) does not take account of biological data.

Mammography detects cancers when they have become established, a significant percentage of which have metastasised.

Mammography is uninformative in the majority of younger women.

Most women with a strong family history suggestive of BRCA1 and 2 are at most risk of developing breast cancer before the age of 50 years.

The investigations lack sensitivity in measuring the extent of the disease.

Some tests (e.g. ultrasound) are operator dependent and therefore the reproducibility is variable.

The investigations are time consuming and costly.

Potential benefits of using serum tumour markers to detect early breast cancer would be in the following areas:

Clinical Care

Clinician

One benefit for the clinician will be the earlier detection of disease. The panel of markers could be used to alert clinicians for example to biopsy small lesions on mammograms which radiographically do not appear suspicious or to an early repeat mammogram or ordering of alternative more sensitive tests such as an MRI or PET scan. The marker measurements themselves should be more objective and reproducible within the limits of variation of the assays compared to current imaging tests.

Patient

The benefits described above in favour of serum markers should result in direct clinical benefits to the patients. Furthermore, at present patients have to wait days to a few weeks for investigational tests. Serum tumour markers could be measured the same day. With an automated system results could even be available to the patients at the same clinic visit, standardisation of the follow-up of at risk patients using a panel of reproducible assays should reduce variation between cancer centres.

Health Care Providers

Health care providers will be able to plan the cost of regular, standardised follow-up of ‘at risk’ individuals and breast cancer patients. The potential cost-benefit of serum markers over radiological imaging in advanced disease has already been estimated. The cost of sequential measurements of serum tumour markers should also be much less than the costs for imaging tests currently carried out in the follow-up of patients with primary disease or women attending the Family History/‘At Risk’ clinics. Furthermore, once proven, such a method of follow-up by serum markers could be carried out by the primary care physician with patients being referred back when the markers become elevated. Such a system of follow-up offers the potential of large cost-savings.

Detection of Recurrence

As indicated above, the use of auto-antibody detection to monitor for disease recurrence offers potential advantages in terms of sensitivity and therefore improved lead time. Aberrant proteins expressed by the primary tumour are also likely to be present in both locally recurring tumours as well as distant metastases. The reappearance of any aberrant proteins which elicited a primary immune response in the patient, will act as a representation of antigen, leading to a rapid secondary immune response. Thus, the presence of minimal amounts of antigen bearing tumour will be amplified many fold by the resultant auto-antibody production. Pilot data on sequential serum samples from patients with recurrent and non-recurrent disease supports this concept.

Benefits

i) Earlier detection of recurrent disease will allow treatment regimes (local or systemic) to be initiated or altered earlier in the course of the disease.

ii) There is already pilot data that such an early intervention strategy based on established markers of tumour bulk (i.e. CA15.3 and CA27.29) lead to at least a longer metastases free interval.

iii) Improved quality of life and potentially improved patient survival of the magnitude seen with adjuvant systemic therapy.

iv) Standardisation of breast cancer follow-up investigations. Currently this varies from clinical examination alone to rigorous imaging assessment (e.g. regular mammography, bone scans, CT scans and blood tests).

Clinical Care

Clinician

Objectivity and reproducibility of the tests would be valuable compared to imaging tests.

Currently, with late diagnosis of metastatic disease, there is only time in some patients to try one systemic therapy, which may or may not be effective. Earlier diagnosis of recurrence would allow clinicians more time to identify and deliver an effective regimen.

Health Care Providers

Advantages to health care workers are similar to those encountered for the ‘at risk’ population. Follow-up assays could be performed routinely in the primary setting, thus decreasing the number of hospital consultations required. Again, such a system of follow-up offers the potential of large cost-savings.

Bladder Cancer

As with breast cancer, a group of 80 patients with bladder cancer was retrospectively analysed using the assays of the invention for the detection of auto-antibodies to MUC1, p53, c-erbB2 and c-myc. Data analysis for this group revealed a detection sensitivity of 80%, though this includes late stage as well as early stage disease.

Specifically, in this group of patients the rate of detection using all four assays was 80%. In the corresponding ‘benign’ group, an apparently high rate of false negatives was noted, 7 positives out of 10 samples. Further assessment of the clinical history of these 7 patients, however, revealed that 6 of them, although benign as far as their urology was concerned (i.e. they did not have a urological malignancy) had either a diagnosis of another cancer (lung, skin cancer, adenocarcinoma of unknown primary), or evidence of neoplasia (colonic polyps, ovarian cysts and pleural effusion).

Colorectal Cancer

The assays of the present invention have been tested for diagnosis of colorectal cancer.

Colorectal cancer ranks as the third leading cause of cancer death in the United States, with in the region of 50,000 deaths and 150,000 new cases per year. The current lifetime risk for an American developing colorectal cancer is approximately 5%. As with a number of other cancers, the risk of developing colorectal cancer increases with age and is rare in those under 40 years of age (3% of cases). A number of risk factors are associated with the development of colorectal cancer, including advancing age; inherited familial adenomatous polyposis; personal or family history of colorectal cancer; personal history of cancer of the ovary, endometrium, or breast; chronic ulcerative colitis or Crohn's colitis. These allow either a population based screening approach or more frequent screening of targeted groups identified to be at increased risk.

Two screening modalities are currently available for colorectal cancer—Fecal Occult Blood Test (FOBT) and Sigmoidoscopy/colonoscopy. Studies have shown that for people aged between 50-80, regular screening by sigmoidoscopy/colonoscopy or repeated fecal occult blood tests (every 1 to 2 years) decrease the number of deaths due to colorectal cancer, but both methods have problems either with cost (sigmoidoscopy/colonoscopy requires clinician time), specificity (FOBT has a specificity of approximately 60%) or willing patient participation. Nevertheless, in the region of 185 million tests are preformed annually and this figure is expected to rise at a rate between 10-15%. Another test currently used by some clinicians in colorectal cancer is a test for the blood borne tumour marker CEA. As with breast and ovarian cancer, this tumour marker is associated with advanced disease and may be used for monitoring disease progression and response to treatment, however, it is not sensitive enough for use as a blood based screening test. New research is suggesting that the detection of nucleic acid coding for aberrant ras protein may be suitable as an early diagnostic tool, but the current sensitivity of the method is only approximately 50% and this is unlikely to improve since not every colorectal cancer displays a mutated ras gene.

The original panel of four auto-antibodies (MUC1, p53, c-erbB2, c-myc) plus the k-ras has been used on a series of 49 patients with colorectal cancers, 21 with polyps and 28 individuals with no evidence of the disease. Data from this study gave a detection rate of 71% for patients with colorectal cancer, 8% for patients with polyps and 3% for patients with no evidence of disease, with a confidence level of 95%. Without the inclusion of the ras assay, a detection rate of less than 50% would have been achieved, therefore confirming the need for specific assays to be included in a panel.

A recent addition to the panel of auto-antibody assays has been one for the detection of auto-antibodies to a truncated APC protein. Pilot data (using a subset of the above series) for the inclusion/non-inclusion of the APC assay into a colon specific panel had demonstrated the following detection rates at a confidence level of 95%:

with APC—75% for patients with colorectal cancer, with no APC detection in normals or polyps.

without APC—60% for patients with colorectal cancer.

This result again emphasises the value of the panel approach.

Prostate Cancer

Using the original panel (MUC1, p53, c-erbB2, c-myc) a detection rate of prostate cancer was increased to 81%. This provides further evidence in support of both the concept of a panel of auto-antibody assays for sensitive cancer detection, and the need for ‘individualising’ the panel of assays to the cancer under investigation.

Ovarian Cancer

Ovarian cancer is generally discovered either by chance or late in the course of the disease when symptoms have become apparent. However, women at increased risk of developing this disease can be identified. For example, women with a first degree relative with ovarian cancer or who have had their first child late in life are at greater risk of developing this form of cancer. Also patients with a strong Family History of Breast Cancer and known to carry a BRCA1 or BRCA2 mutation are also at increased risk of developing ovarian cancer. Future trends in western society—i.e. an increasingly elderly population and older age at first pregnancy—are likely to result in an increase in the incidence of this disease. However, since early disease rarely produces symptoms, patients still tend to be diagnosed with advanced symptomatic disease. Current early diagnosis of the disease relies on chance detection of abnormality upon physical examination, followed by a number of time consuming, complex and expensive confirmatory tests such as ultrasound, X-ray, paracentesis (if any fluid has collected in the abdomen), body scans (either CT or MRI), laparoscopy. The blood borne tumour antigen marker CA125 may also be used to aid in diagnosis of advanced disease, with raised levels of this marker being suggestive of ovarian cancer. However, it has neither the sensitivity nor specificity to be recommended as a screening test.

A major concern with ovarian cancer is its readiness to spread from the original site to secondary sites such as the brain, bone and bowels. Early detection of the disease is therefore a major contributor to a successful treatment outcome and so is highly desirable. However, diagnosis is not simple in this particular form of cancer and currently no regular screening programme is available at economic cost. There is therefore scope for the development of blood borne tumour markers for use earlier in the disease course and for introduction into a screening modality.

MUC1 mucins are already of clinical value in advanced breast cancer but they are as yet of no value in the primary disease due to the insensitivity of the current assays. Similarly, as mentioned above, the mucin CA125 is also of use in advanced ovarian cancer. As with MUC1 mucin, the glycosylation of CA125 is aberrant leading to the production of cryptic epitopes capable of eliciting an immune response in the host. However, the same basic methodology for an assay for the detection of auto-antibodies to CA125 as that adopted for MUC1 mucin can be applied.

The data disclosed herein validates the utility of a panel of auto-antibody assays in the detection of neoplasia in a number of different cancers. They also confirm the requirement for specific panels in the detection of specific cancers. Furthermore, they highlight the value of tissue specific markers particularly in a subset of patients known to at high risk of developing breast or ovarian cancer due to BRCA1 and/or BRCA2 mutations. Indeed although the data given above relates to specific panels, it will be readily apparent that the panel of tumour marker antigens may be tailored to particular applications. A panel of at least p53 and c-erbB2 is particularly useful for many types of cancer and can optionally be supplemented with other markers having a known association with the particular cancer, or a stage of the particular cancer, to be detected. For example for breast cancer the panel might include MUC1 and/or c-myc and/or BRCA1 and/or BRCA2 and/or PSA whereas bladder cancer the panel might optionally include MUC1 and/or c-myc, for colorectal cancer ras and/or APC, for prostate cancer PSA and/or BRCA1 and/or BRCA2 or for ovarian cancer BRCA1 and/or BRCA2 and/or CA125. For hepatocellular carcinoma alphafetoprotein (αFP) and/or p62, for lung cancer annexing I and/or annexin II. There are other preferred embodiments in which p53 or c-erbB2 are not necessarily essential. For example, in the case of breast cancer suitable panels may be selected from the following:

p53 and MUC1 with optional c-erbB2 and/or c-myc, and/or BRCA1 and/or BRCA2 and/or PSA;

p53 and c-myc with optional c-erbB2 and/or MUC1 and/or BRCA1 and/or BRCA2 and/or PSA;

p53 and BRCA1 with optional c-erB2 and/or MUC1 and/or c-myc and/or BRCA2 and/or PSA;

p53 and BRCA2 with optional c-erbB2 and/or MUC1 and/or c-myc and/or BRCA1 and/or PSA;

c-erbB2 and MUC1 with optional p53 and/or c-myc, and/or BRCA1 and/or BRCA2 and/or PSA;

c-erbB2 and c-myc with optional p53 and/or MUC1 and/or BRCA1 and/or BRCA2 and/or PSA;

c-erbB2 and BRCA1 with optional p53 and/or MUC1 and/or c-myc and/or BRCA2 and/or PSA;

c-erbB2 and BRCA2 with optional p53 and/or MUC1 and/or c-myc and/or BRCA1 and/or PSA;

In the case of colorectal cancer suitable panels could be selected from the following:

p53 and ras with optional c-erbB2 and/or APC;

p53 and APC with optional c-erbB2 and/or Ras;

Ras and APC with optional p53 and/or c-erbB2

In the case of prostate cancer suitable panels could be selected from the following:

p53 and PSA with optional BRCA1 and/or c-erbB2;

c-erbB2 and PSA with optional p53 and/or BRCA1.

In the case of ovarian cancer suitable panels could be selected from the following:

p53 and CA125 with optional c-erbB2 and/or BRCA1;

c-erbB2 and CA125 with optional p53 and/or BRCA1.

Other possible markers for the panel are shown in Tables A and B below.

Table A

Additional auto-antibodies to human cancer antigens which, when quantitated individually or in combination with auto-antibodies to other antigens (Diagnostic Test Panel), are useful in cancer diagnosis

| Breast | Prostate | Ovarian | Colorectal |
|---|---|---|---|
| Cancer | Cancer | Cancer | Cancer |
| CEA gene | CEA gene | CEA gene | CEA gene |
| family | family | family | family |
| members | members | members | members |
| PTH-RP | PTH-RP | PTH-RP | PTH-RP |

TABLE B
Auto-antibodies to human cancer antigens whose induction and epitopic specificity are clinically relevant in
the diagnosis and treatment of cancer.

| Breast | Prostate | Ovarian | Gastric | Lung | Colorectal | Pancreatic | Cervical | | Liver |
|---|---|---|---|---|---|---|---|---|---|
| Cancer | Cancer | Cancer | Cancer | Cancer | Cancer | Cancer | Cancer | General | Cancer |
| p53 | p53 | p53 | CEA gene | CEA gene | p53 | CEA gene | p53 | TK | p53 |
| | | | family | family | | family | | | |
| | | | members | members | | members | | | |
| c-erbB2 | c-erbB2 | c-erbB2 | Pro-gastrin | p53 | c-erbB2 | p53 | c-erbB2 | PTH-RP | c-erbB2 |
| c-myc | PSA | BRCA1 | Gastrin | c-erbB2 | ras | CA19-9 | SCC | | αFP |
| | | | G17 | | | | | | |
| MUC1 | BRCA1 | CA125 | Gastrin | CYFRA | APC | c-erbB2 | HPV | | p62 |
| | | | G34 | 21-1 | | | sub-types | | |
| BRCA1 | Kallikrein | PTH-RP | CA19-9 | PHT-RP | CEA gene | CA72-4 | | | |
| | | | | | family | | | | |
| | | | | | members | | | | |
| BRCA2 | PTH-RP | | CA72-4 | Vaso- | Pro-gastrin | | | | |
| | | | | pressin | | | | | |
| PSA | | | p53 | Gastrin | Gastrin | | | | |
| | | | | releasing | G17 | | | | |
| | | | | peptide | | | | | |
| CEA gene | | | | Annexins I | Gastrin G34 | | | | |
| family | | | | and II | | | | | |
| members | | | | | | | | | |
| CYFRA 21-1 | | | | Hu | PTH-RP | | | | |
| PTH-RP | | | | KOC | | | | | |

In another aspect the present invention provides compositions and methods of determining the immune response of a patient to two or more circulating tumour marker proteins or to tumour cells expressing said tumour marker proteins and identifying which one of said two or more tumour marker proteins elicits the strongest immune response in the patient, the method comprising contacting a sample of bodily fluids from said patient with a panel of two or more distinct tumour marker antigens, measuring the amount of complexes formed by binding of each of said tumour marker antigens to autoantibodies present in the sample of bodily fluids, said autoantibodies being immunologically specific to said tumour marker proteins and using the measurement obtained as an indicator of the relative strength of the immune response to each tumour marker protein and thereby identifying which one of said two or more tumour marker proteins elicits the strongest immune response in the patient.

The assays described above, which may be hereinafter referred to as a ‘selection assay’ are useful in the selection of a course of vaccine treatment wherein the single tumour marker protein identified as eliciting the strongest immune response or a combination of markers eliciting strong immune response is/are used as the basis of an anti-cancer vaccine treatment.

Individual patients produce individual immune response profiles. In other words, immune responses to different antigens are generated on an individual basis. This may be due to differences in the individuals immuno-tolerance to tumour associated proteins, or it may be due to individualised expression patterns of tumour associated proteins. Thus, vaccination protocols that concentrate on delivering a standard vaccine to every patient are likely to fail for a large proportion of patients due partially to the lack of expression of that particular tumour associated protein by individual tumours. It is therefore envisaged that a more appropriate approach would be to assess an individual's immune response profile by the use of auto-antibody assays. This would then define both the tumour associated proteins that have elicited an immune response in that individual, and the most immunogenic of those proteins in that individual. A decision could then be made, based on biological information, regarding the most suitable vaccination programme for that individual.

Preferred tumour marker antigens for use in the selection profile assay are any of the tumour marker antigens mentioned above and preferably the antigens are labelled with biotin. The actual steps of detecting autoantibodies in a sample of bodily fluids may be performed in accordance with known immunological assay techniques, as described above for the panel assay.

The invention also provides methods for the detection or quantitative measurement of the immune response of a mammal to a circulating tumour marker protein or tumour cells expressing the tumour marker protein wherein the tumour marker protein is MUC1, c-erbB2, Ras, c-myc, BRCA1, BRCA2, PSA, APC, CA125 or p53, PTH-RP, CYRFA 21-1, kallikrein, pro-gastrin, gastrin G 17, gastrin G34, CA19.9, CA72.4, gastrin releasing peptide, SCC, TK, αFP, p62, annexins I and II, Hu, KOC or any of the CEA gene family members, or an antigen of HPV, preferably a sub-type associated with cervical cancer risk, the method comprising the steps of contacting a sample of bodily fluids from the mammal with the tumour marker antigen and determining the presence or absence of complexes of the tumour marker antigen bound to autoantibodies immunologically specific to the tumour marker protein or antigenic fragment thereof, whereby the presence of said complexes is indicative of the immune response to said circulating tumour marker protein or tumour cells expressing the tumour marker protein.

The assays described above, which may be hereinafter referred to as ‘single marker assays’, use a single type of tumour marker as antigen rather than using a panel of two or more tumour markers. The single marker assays may be used in any clinical situation, for example, screening for early neoplastic or carcinogenic change in asymptomatic patients, identification of individuals ‘at risk’ of developing cancer, early diagnosis and early detection of recurrence in a patient previously diagnosed as carrying tumour cells which patient has undergone treatment to reduce the number of said tumour cells or in predicting the response of a patient to a course of anti-cancer treatment, including surgery, radiotherapy, immune therapy, vaccination etc.

The single marker assays are particularly useful in situations where the tumour marker eliciting the strongest immune response in a given patient has been previously identified, possibly using the selection assay described above. For example, in a situation in which an initial selection assay has been performed to establish which tumour marker elicits the strongest immune response in a given patient, subsequent follow-up, detection of recurrence or monitoring of treatment may be carried out using a single marker assay to only detect or measure autoantibodies to that tumour marker previously identified as eliciting a strong immune response in that patient.

The actual steps of detecting autoantibodies in a sample of bodily fluids may be performed in accordance with known immunological assay techniques, as described above for the panel assay. Preferably the tumour marker protein used as antigen is labelled with biotin so that it may be easily attached to a solid support by means of the biotin/avidin or biotin/streptavidin interaction.

In a further aspect, the present invention provides a preparation comprising a human MUC1 protein which MUC1 protein manifests all the antigenic characteristics of a MUC1 protein obtainable from the bodily fluids of a patient with advanced breast cancer.

Preferably the MUC1 protein exhibits altered affinity for the antibodies B55, C595, BC4W154, DF3, B27.29, 115D8, 27.1, SM3, Ma552, HMPV and BC2 compared to MUC1 protein isolated from normal human urine. Most preferably the MUC1 protein is isolated from the serum of one or more human patients with advanced breast cancer. This can be accomplished using the protocol given in the Examples listed herein.

As will be described in detail in Example 2, there are immunological differences between MUC1 isolated from normal individuals and MUC1 isolated from patients with advanced breast cancer. Possibly as a result of these differences, the MUC1 protein isolated from serum of patients with advanced breast cancer (hereinafter referred to as ABC MUC1) is more sensitive when used as antigen in an assay to detect autoantibodies specific to MUC1 than either MUC1 isolated from urine of normal individuals, synthetic MUC1 or MUC1 isolated from a range of different cultured cells. MUC1 isolated from the serum of patients with advanced breast cancer is therefore preferred for use as antigen in the panel assay method and the single marker assay methods described herein.

MUC1 has recently attracted interest as a target for immunotherapy of adenocarcinomas and several Phase I clinical trials involving different MUC1 vaccine substrates, adjuvants and carrier proteins have been carried out (Goydos, J. S. et al. (1996) J Surgical Res. 63: 298-304; Xing, P. X. et al. (1995) Int. J Oncol. 6: 1283-1289; Reddish, M. A. et al. (1996) Cancer Immunol. Immunother. 42: 303-309; Graham, R. A. et al. (1996) Cancer Immunol. Immunother. 42: 71-80). Methods for the detection of anti-MUC1 autoantibodies using MUC1 isolated from the serum of patients with advanced breast cancer as antigen will be of particular use in monitoring the success of MUC1 vaccine therapy. In this case the aim of the assay will be to detect anti-MUC1 antibodies produced in response to the vaccine rather than autoantibodies i.e. antibodies produced in response to an exogenous antigen introduced into the body by vaccination. Methods for the detection of antibodies directed to other tumour markers would also be of use in monitoring the success of vaccine therapy using the relevant tumour marker. For example, following vaccination with a p53 antigenic preparation, the presence of anti-p53 antibodies could be monitored using the assay based on the use of biotinylated p53 antigen described in the examples given below. Moreover, the panel assay method could also be used in monitoring the success of vaccine therapy, for example, in a situation where an individual has been vaccinated with an antigenic preparation designed to elicit antibodies to two or more different tumour markers. Another example would be where an individual had been vaccinated (with an antigenic preparation designed to elicit antibodies to one or more tumour markers). The panel assay method could be used to predict those likely to benefit or not and/or to monitor for subsequent rising antibody levels (either to the markers in the antigen's preparation or other markers) as measures/indicators that the tumour was developing and/or progressing despite the vaccination strategy.

In a still further aspect the present invention provides a method of detecting recurrent disease in a patient previously diagnosed as carrying tumour cells, which patient has undergone treatment to reduce the number of said tumour cells, which method comprises steps of contacting a sample of bodily fluids from the patient with MUC1 protein or an antigenic fragment thereof, determining the presence or absence of complexes of said MUC1 protein or antigenic fragment thereof bound to autoantibodies present in said sample of bodily fluids, said autoantibodies being immunologically specific to MUC1, whereby the presence of said complexes indicates the presence of recurrent disease in said patient.

The method described above may be repeated on a number of occasions to provide continued monitoring for recurrence of disease. The method is particularly preferred for the monitoring of patients previously diagnosed with primary breast cancer, colorectal cancer, prostate cancer, bladder cancer, liver, lung, pancreatic, ovarian, gastric, endometrial or cervical cancers, which patients have undergone treatment (e.g. surgery) to remove or reduce the size of their tumour. In this instance, the presence of anti-MUC1 autoantibodies in the patient's serum after treatment may be indicative of recurrence of disease.

Also provided by the invention are assay kits suitable for performing the methods for the detection of autoantibodies described herein. Such kits include, at least, samples of the tumour marker antigens to be used as antigen in the assay and means for contacting the sample to be tested with a sample of the antigen.

The invention as discussed above relates to detection in a patient sample of bodily fluid of antibodies or autoantibodies to a cancer-associated antigen.

As already extensively discussed herein cancer markers often differ from the corresponding wild-type proteins in such a way that they are recognised as foreign molecules by the immune system of an individual, triggering an autoimmune-response. For example, modified forms of p53, MUC-1, c-myc, c-erb3 and Ras proteins elicit production of autoantibodies as aforesaid.

As will be described in the Examples below, that autoantibodies produced by patients suffering from cancer specifically recognise cancer-associated marker proteins from the same patients or from other patients with cancer and show very low cross-reactivity with wild-type forms of these proteins in a non-cancer population. Furthermore, the above autoantibodies have a much higher sensitivity than the antibodies currently used in routine tests and are therefore able to detect smaller quantities of cancer-associated marker proteins. Autoantibodies produced by patients with cancer can be used to design alternative, more reliable and sensitive tests to detect pre-neoplastic or carcinogenic modifications in an individual from the initial occurence. These assays may also be employed to detect cancer or pre-neoplasia in any other mammal, by utilising autoantibodies produced by a mammal from the same species as the one to be tested or autoantibodies having the same characteristics as such.

Such assays provide a more sensitive and specific assay system for the detection of pre-neoplasia or cancer in a mammal, which allows the detection of cancer-associated marker proteins from the early stages of the disease.

Accordingly, in a further aspect the invention provides an in vitro method for detecting a cancer-associated marker protein present in a bodily fluid of a mammal which method comprises the steps of:

(a) contacting a sample of bodily fluid from said mammal with antibodies directed against at least one epitope of said marker protein; and

(b) detecting the presence of any complexes formed between said antibodies and any marker protein present in said sample;

wherein said antibodies are mammalian autoantibodies to said cancer-associated marker protein which are derived from the same species as the mammal from which said sample has been obtained.

The presence of said complexes is indicative of the presence of cancer associated marker proteins in said mammal.

In this context “derived” means an autoantibody or autoantibodies isolated from the said species or an autoantibody or autoantibodies having the characteristics of an autoantibody or autoantibodies isolated from said species.

Further, in this aspect of the invention the term “autoantibody” refers not only to an antibody directed against a self-originating antigen, which antibody is naturally occurring in the circulation of an individual but also to an antibody which exhibits the characteristics of the naturally occurring antibody in that it recognises the said self-originated antigen but which is produced outside the body, for example, by an immortalised cell.

The methods of the invention may employ a single autoantibody directed against a particular cancer marker protein. Alternatively, a panel of autoantibodies recognising a number of cancer-associated proteins may be utilised in order to obtain a profile of cancer markers present in a particular individual. This leads to a more reliable diagnosis and provides information useful in the choice of the most appropriate treatment for an individual.

The assay methods of the invention are performed on a sample of a biological fluid from the patient such as, for example, plasma, serum, whole blood, urine, lymph, faeces, cerebrospinal fluid or nipple aspirate, depending of the nature of the cancer to be detected. Since it is non-invasive the assay can be repeated as often as it is necessary to screen for early neoplastic or carcinogenic modifications, to follow the development of the disease, to test for recurrence of the disease, to verify the efficacy of a treatment or to select the most appropriate treatment for a particular patient.

The methods of the invention can be performed using any immunological technique known to those skilled in the art of immunochemistry. As examples, ELISA, radio immunoassays or similar techniques may be utilised. In general, an appropriate autoantibody is immobilised on a solid surface and the sample to be tested is brought into contact with the autoantibody. If the cancer marker protein recognised by the autoantibody is present in the sample, a complex autoantibody-marker is formed. The complex can then be directed or quantitatively measured using, for example, a labelled secondary antibody which specifically recognises an epitope of the marker protein. The secondary antibody may be labelled with biochemical markers such as, for example, horseradish peroxidase (HRP) or alkaline phosphatase (AP), and detection of the complex can be achieved by the addition of a substrate for the enzyme which generates a colorimetric, chemiluminescent or fluorescent product. Alternatively, the presence of the complex may be determined by the addition of a marker protein labelled with a detectable label, for example an appropriate enzyme. In this case, the amount of enzymatic activity measured is inversely proportional to the quantity of complex formed and a negative control is needed as a reference to determine the presence of antigen in the sample. Another method for detecting the complex may utilise antibodies or antigens that have been labelled with radioisotopes followed by measure of radioactivity.

The methods of the present invention can be performed in a qualitative format, which determines the presence or absence of a cancer marker protein in the sample or in a quantitative format, which, in addition, provides a measurement of the quantity of cancer marker protein present in the sample. The quantity of marker protein present in a sample may be calculated utilising any of the above described techniques. In this case, prior to performing the assay, it is necessary to draw a standard curve by measuring the signal obtained, using the same detection reaction that will be used for the assay, from a series of standard samples containing known concentrations of the cancer marker protein. The quantity of cancer marker present in a sample to be screened is then interpolated from the standard curve.

If it is necessary to verify the presence of a number of cancer marker proteins in a sample, the assay of invention may be performed in a multi-well assay plate where each of the different autoantibodies utilised is placed in a different well. Alternatively, multiple antigens may be placed in a single well or may be arranged in the form of an array.

The methods of the invention can be employed in a variety of clinical situations such as, for example, in the assessment of the predisposition of an individual towards the development of a cancer, in the detection of pre-neoplastic or carcinogenic modifications in asymptomatic patients, in the diagnosis of primary or secondary cancer, in monitoring the progression of the disease in a patient, in screening for recurrence of carcinogenic modifications in a patient who has previously been diagnosed as carrying cancer cells and has undergone a therapy to reduce the number of these cells or in the choice of the more appropriate anti-cancer treatment for a patient suffering from cancer. The methods of the invention are also suitable for veterinary use in the same clinical situations as the ones described above.

The assay methods of the invention may be employed to detect cancer marker proteins that are associated with a variety of cancers such as, for example, lymphomas, leukaemia, breast cancers, colorectal cancers, lung cancers, pancreatic cancers, prostate cancers, cervical cancers, ovarian cancers, endometrial cancers, liver cancers and cancers of the skin. The methods of the invention are particularly suitable to detect and monitor primary cancer and advanced cancer particularly primary breast cancer (PBC) and advanced breast cancer (ABC).

In a further aspect the invention provides autoantibodies and reagents comprising said autoantibodies for use in the assay, which specifically recognise at least one epitope of a mammalian cancer-associated marker protein. Such autoantibodies may be isolated from the blood or peripheral blood monocytes of such a mammal, preferably a human. Alternatively, the autoantibodies can be produced by immortalised B lymphocytes and directed to an antigen originated in the mammal itself. The reagents comprising autoantibodies according to this aspect of the invention are particularly suitable for use in the detection of mammalian cancer-associated marker proteins in body fluids. Preferred autoantibodies to use in the assay include those against cancer-associated forms of the glycoprotein MUC1 (Batra, S K. et al. (1992) Int J. Pancreatology 12: 271-283), the signal transduction/cell cycle regulatory protein c-myc (Blackwood, E. M. et al. (1994) Molecular Biology of the Cell 5: 597-609), p53 (Matlashewski, G. et al. (1984) EMBO J. 3: 3257-3262), c-erb□2 (Dsouza, B. et al. (1993) Oncogene 8: 1797-1806) and Ras (Gnudi, L. et al. (1997) Mol. Endocrinol. 11: 67-76). However, autoantibodies against any other cancer-associated marker protein may be employed in the assay. Particularly suitable for the detection of breast cancers are autoantibodies against a modified MUC1, BRCA1, BRCA2, p53, c-myc,c-erb□2 or Ras protein associated with primary breast cancer and autoantibodies against a modified MUC1, BRCA1, BRCA2 p53, c-myc,cerb□2 or Ras protein associated with advanced breast cancer. Auto-antibodies directed against any of antigens for any of the types of cancer set out in Tables A and B above are also suitable for use in this respect of the invention. These autoantibodies are preferably derived from patients diagnosed with the same type of cancer as the one to which these cancer marker proteins are associated.

The invention also provides immortalised cell populations capable of producing the above autoantibodies.

The cell populations of the invention may be produced by any method known in the art. As will be described in detail in Example 17 below, B cells from patients diagnosed with cancer may be, for example, immortalised with Epstein Barr Virus. ELISA or any similar techniques may be performed to screen for the production of autoantibodies, utilising marker proteins obtained from a patient affected from cancer which have been immobilised on a solid support.

The invention further provides kits for detecting one or more cancer-associated marker proteins in the biological fluids of a mammal. Such kits include at least mammalian autoantibodies directed against one or more epitopes of a cancer-associated marker protein and means for detecting the formation of complexes between the autoantibodies and the cancer-associated marker protein. Preferably, the autoantibodies are immobilised on a solid surface.

In another of its aspects the present invention relates to a method of treating cancer by administering to a patient an effective amount of auto-antibody to a cancer-associated antigen. The antibody may exhibit its anti-cancer effect by virtue of its own inherent properties, or as a passive vaccine or may have attached reversibly or otherwise, a therapeutic agent (e.g cytotoxic drug, cellular toxin, enzyme inhibitor).

In yet a further aspect the invention relates to a diagnostic imaging technique which comprises administering to a patient an auto-antibody directed against a cancer-associated antigen wherein said auto-antibody is attached to an imaging agent.

The invention also relates to pharmaceutical composition comprising an auto-antibody and a pharmaceutically acceptable carrier or diluent, an anti-cancer vaccine comprising an auto-antibody and a pharmaceutically acceptable carrier or diluent and an auto-antibody preparation comprising said auto-antibody attached, reversibly or otherwise, to a therapeutic agent e.g. cytotoxic agent or an imaging agent.

The auto-antibody for in vivo use in the aspect of the invention may be directed against any one of the cancer-associated antigens specifically identified herein. Furthermore, the auto-antibody preparation, pharmaceutical composition or vaccine may include auto-antibodies directed to more than one of the cancer-associated antigens identified herewith in any combination.

The therapeutic, prophylactic or in vivo diagnostic methods described above may be in respect of any of the cancers herein described as well as for use in asymptomatic or ‘at risk’ patients.

The pharmaceutical composition, vaccine or auto-antibody preparation may be formulated with a suitable carrier or diluent as it is well-known in the pharmaceutical arts. Such formulations can include in addition to antibody, a physiologically acceptable diluent or carrier possibly in a mixture with other agents such as other antibodies. Suitable carriers include but are not limited to physiological saline, phosphate buffered saline, phosphate buffered saline glucose and buffered saline. Alternatively, the auto-antibody may be lyophilized and reconstituted for use when needed by the addition of an aqueous buffered solution as described above. The antibody pharmaceutical composition may be administered by the intravenous, intramuscular subcutaneous and intraperitoned route.

The dosages of such antibodies in pharmaceutical compositions will vary with the compositions being treated and the recipient of the treatment. Ranges of 1 to 100 mg per day, preferably 1-10 mg, can be contemplated.

The auto-antibodies of the invention may be used in a form of individualised cancer management.

Current research world wide is demonstrating the potential utility of a number of immuno techniques in cancer management such as:

Immunoscinitgraphy, both for detection of occult disease and for the localisation of unknown primary tumours;

Immunotargeting, the use of murine chimeric or humanised antibodies for the delivery of cytotoxic therapies direct to the tumour;

Sentinal node location using radiolabelled antibodies.

The ability to purify auto-antibodies from a sample of patient sera provides an opportunity to use the patient's own anti-tumour antibodies for these techniques. An alternative apparently would be to use anti-tumour antibodies from one or more patients with the same tumour type as the individual to be tested. These may be autoantibodies derived from patients sera or antibodies having the characteristics of an antibody or antibodies derived from patients sera. A typical management plan could be as follows:

Patient presents for breast cancer screening (for instance),

Blood sample taken and tested for anti-tumour immuno profile;

Profiling indicates auto-antibodies to a number of tumour associated proteins—confirmation of cancer;

Further larger blood sample taken (?250 ml?) before patient goes home;

Antibodies purified, aliquot labelled with gamma emitting radiolabel, rest stored;

Patient returns for pre-operative check-up and immunoscintigraphy using an aliquot of their radiolabelled antibodies. Since antibodies are the patients own, no risk of human anti-mouse immune reaction (HAMA) as is seen when mouse antibodies are used;

Clinical assessment of the stage (i.e. nodal involvement, occult metastases, distant spread), of the cancer prior to surgery using the gamma image produced by the immunoscintigraphy. Again, no risk of HAMA from repeated antibody dose;

At surgery, (after clearance of immunoscintigraphy antibody dose), radiolabelled antibody injected into tumour vasculature for localisation into sentinal node. Again, no risk of HAMA from repeated antibody dose. Sentinal node can then be confirmed by hand held gamma detector and removed for histological assessment.

| | | |
|---|---|---|
| Patient enters | Patient monitored | Immunotargeted |
| individualised | for disease | therapy commences |
| vaccination | progression using | using patients |
| programme based | auto-antibody | own antibodies. |
| on immuno profile | | Again, no HAMA. |
| assays | | |

Thus, a complete cancer management package can be tailored to match each individual patient's cancer. As noted above, in future it may be possible to access a source of such antibodies which can be used in individual patients without having to do this from the blood of each patient.

Some MUC1 peptide vaccines currently in clinical trials do not produce antibodies which recognise tumour associated MUC1 antigen, rather they produce antibodies that recognise naked MUC1 peptide. It is believed that problems with the recognition of tumour associated proteins by vaccine induced antibodies stem from the assumption that those regions of molecules that appear to be immunodominant in mice are not necessarily also immunodominant in humans. Thus the hosts immune response to tumour, is a new approach to vaccine development.

Having developed antigens for use in ELISA's for the detection of auto-antibodies, those antigens or epitopes of such antigens may be utilised as vaccines in their own right.

Therefore, in another of its aspects the invention relates to a method of vaccinating an individual comprising administering to said individual an effective amount of a cancer-associated antigen comprising a specific cancer-associated epitope. The invention also relates to a vaccine comprising said cancer-associated antigen or a cancer-specific epitope thereof and a pharmaceutically acceptable carrier or diluent.

The vaccine of this aspect of the invention may comprise any one or more of the specific cancer-associated antigens identified herein in any combination. The vaccine may be in respect of any of the cancers identified herein. Suitable carriers and diluents for formulating the vaccine are known in the art.

The following protocol may be used for the development of such vaccines.

Antigens developed for use in ELISAs for detection of auto-antibodies may be utilised for the purification of auto-antibodies from patient sera by immunochromatography. The assays of the present invention have been able to demonstrate this with regard to the purification of circulating auto-antibodies to MUC1, p53, c-myc and c-erbB2 from sera from patients with advanced breast cancer. By having purified auto-antibodies, the epitopes which induced the antibodies can be characterised and elucidated using various epitope mapping techniques. By combining information gained from this assessment of auto-antibodies with molecular modelling information, it is possible to determine those regions of mutated proteins that are immunogenic (for instance hydrophilic and external turn regions). Fragments containing the regions of interest are produced by specific proteolytic cleavage of the whole molecule as well as by PCR amplification of the appropriate region of DNA and expression of the encoded peptide. These fragments are then probed with known positive and negative sera to determine their immunoreactivity. Once immunoreactive fragments have been isolated, these will be further broken down into small synthetically produced peptides in order to more closely define the epitope. It will also be possible to produce phage libraries for panning to determine small epitopes. Once epitopes are defined, their immunogenic capabilities will be ascertained by the in vitro stimulation of human lymphocytes. Any antibodies so induced are tested for their cytotoxic capabilities against human cancer cell lines known to express the appropriate protein.

The contents of all documents, articles and references cited herein are incorporated herein by reference.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting.

The above disclosure generally describes the present invention. A more complete understanding can be obtained by reference to the following examples. These examples are described solely for purposes of illustration and are not intended to limit the scope of the invention. Although specific terms have been employed herein, such terms are intended in a descriptive sense and not for purposes of limitations.

### EXAMPLES

### Example 1

### Isolation of ABC MUC1 from Advanced Breast Cancer Patients

Method

ABC MUC1 was purified from pooled sera taken from 20 patients with advanced breast cancer using immunoaffinity chromatography as follows:

The mouse monoclonal anti-MUC1 antibody B55 (also known as NCRC 11 and described by Ellis et al. (1984) Histopathology. 8: 501-516 and in International patent application No. WO 89/01153) was conjugated to CNBr-sepharose beads. Pooled sera from patients diagnosed with advanced breast cancer was diluted 1/10 in phosphate buffered saline (PBS) and then incubated with the antibody conjugated sepharose beads (25 ml diluted sera to 1 ml packed volume of beads) overnight at 4° C. with rolling. The beads were then packed by centrifugation and the supernatant removed. In order to wash away unbound serum components the beads were resuspended in PBS, rolled for 10 minutes, packed by centrifugation and the supernatant removed. This washing sequence was repeated 5 times (or until A28Onm of the supernatant was ˜0). The washed beads were then resuspended in 0.25M glycine pH 2.5, rolled at room temperature for 10 minutes, packed by centrifugation and the supernatant removed. This supernatant was adjusted to pH 7 by the addition of Tris and stored at 4° C. labelled ‘glycine fraction’. The beads were then resuspended in 1 ml 25 mM diethylamine (DEA) pH 11, rolled at room temperature for 10 minutes, packed by centrifugation and the supernatant removed. This supernatant was again adjusted to pH 7 by the addition of Tris and stored at 4° C. labelled ‘25 DEA fraction’. The beads were finally resuspended in 1 ml 100 mM DEA pH 11, rolled at room temperature for 10 minutes, packed by centrifugation and the supernatant removed. The final supernatant was again adjusted to pH 7 by the addition of Tris and stored at 4° C. labelled ‘100 DEA fraction’. The MUC1 content of the three fractions (glycine fraction, 25 DEA fraction and 100 DEA fraction) was confirmed by ELISA using the mouse monoclonal anti-MUC1 antibody C595 (commercially available from Serotec).

### Example 2

### Immunological Characterisation of ABC MUC1 Isolated from the Serum of Patients with Advanced Breast Cancer

ABC MUC1 isolated from the serum of at least 20 patients with advanced breast cancer according to the procedure described in Example 1 can be distinguished from MUC1 isolated from the urine of normal human subjects (normal human urinary MUC1) on the basis of altered affinity for the following mouse monoclonal anti-MUC1 antibodies:

| | |
|---|---|
| B55 (NCRC 11) | |
| C595 | |
| BC4W154 | Obtainable from Hybritech, Inc |
| DF3 | Obtainable from Centocor |
| B27.29 | Obtainable from Biomira, Inc |
| 115D8 | Obtainable from Centocor |
| 27.1 | Obtainable from Austin Research Institute |
| SM3 | Obtainable from the Imperial Cancer Research Fund |
| Ma552 | Obtainable from CanAg |
| HMPV | Obtainable from Austin Research Institute |
| BC2 | Obtainable from Austin Research Institute |

Normal urinary MUC1 is available from Dr M. R. Price, Cancer Research Laboratories, The University of Nottingham, University Park, Nottingham. NG7 2RD, United Kingdom.

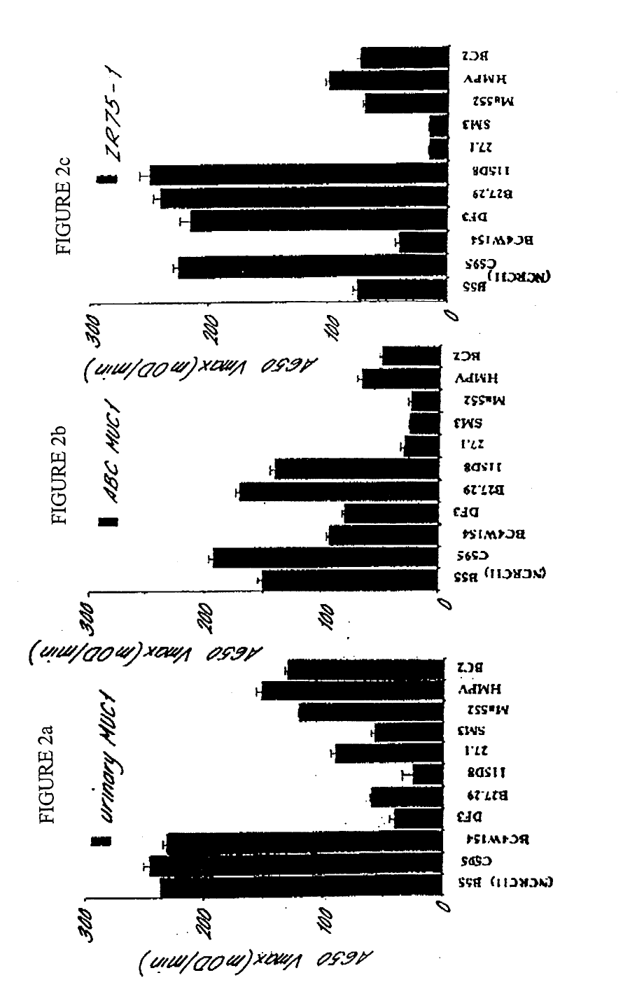

The affinity of each of the above antibodies for ABC MUC1, normal human urinary MUC1 and also MUC1 protein purified from the human breast cancer cell line ZR75-1 (purified from a tissue culture supernatant by gel filtration) was measured by performing calorimetric ELISA assays using each of the different antibodies and secondary anti-immunoglobulin antibodies conjugated to HRP. Following addition of the calorimetric substrate (TMB), measurements were taken of OD at 650 nm. The results of the ELISA assays are presented graphically in FIG. 2. Values of Kd for the binding of several of these antibodies to ABC MUC1 and normal human urinary MUC1 are summarised in Table 1:

TABLE 1
Kd values for binding of monoclonal antibodies
to ABC MUC1 and normal human urinary MUC1.

| Monoclonal | Kd vs ABC MUC1 | Kd vs urinary MUC1 |
|---|---|---|
| BC4W154 | 2.4 × 10⁻⁷ | 1.7 × 10⁻⁹ |
| 115D8 | 1 × 10⁻⁸ | 3.38 × 10⁻⁸ |
| C595 | 2.4 × 10⁻⁸ | 2.5 × 10⁻⁸ |

### Example 3

### Cloning of Biotinylated p53

Method

Commercially available cDNA for p53 (E. coli clone pBH53, deposited in the American Type Culture Collection under accession number 79110) was cloned into the PinPoint™ plasmid vector (Promega Corporation, Madison Wis., USA) using standard molecular biology techniques. The PinPoint™ vector is designed to facilitate the production of fusion proteins comprising a biotinylation domain (consisting of a fragment of a biotin carboxylase carrier protein) fused N-terminally to the target protein of interest. Care was therefore taken during the cloning procedure to ensure that the reading frame of p53 was maintained in the fusion protein. Procedures for cloning in PinPoint™ vectors are described in detail in the Promega Protocols and Applications Guide obtainable from Promega Corporation, Madison Wis., USA.

Fusion proteins expressed from the PinPoint™ vector in E. coli are biotinylated by an enzyme system of the E. coli host cells and may therefore be purified or bound to an assay plate using conventional avidin or streptavidin technology. For example, procedures for purification of the fusion protein using avidin covalently attached to a polymethacrylate resin are described in the Promega Protocols and Applications Guide obtainable from Promega Corporation, Madison Wis., USA.

### Example 4

### Cloning of c-erbB2

Method

Full-length cDNA encoding c-erbB2 was cloned from the human breast cancer cell line ZR75-1, which can be induced to up-regulate c-erbB2 expression by treatment with the anti-cancer drug tamoxifen.

Two T25 flasks of sub-confluent ZR75-1 cells (available from the American Type Culture Collection and from the European Collection of Cell Cultures, deposit number ATCC CRL1500) grown in RPMI plus 10% foetal calf serum were induced to express c-erbB2 by 4 day stimulation with tamoxifen at 7.5 μM (see Warri et al. (1996) Eur. J. Cancer. 32A: 134-140). The cells were then harvested using trypsin/EDTA and washed three times with PBS.

mRNA was extracted from the cell pellet using a Dynabead mRNA purification kit according to the manufacturer's recommended protocol. The mRNA was then used as a template for first strand cDNA synthesis using the Pharmacia Ready-to-go™ T primed first strand cDNA synthesis kit. cDNA/mRNA was then blunt end ligated into the EcoRV site of the PinPoint™ vector. The ligation products were then transformed into Top 10 F E. coli cells (Invitrogen) following the manufacturer's supplied protocol and the transformed cells grown overnight on LB agar plates containing ampicillin. Colonies of the transformed E. coli were copied onto nitrocellulose filter and then grown for 2 hours on LB agar containing ampicillin and IPTG (1 mM). The colonies on the nitrocellulose filter were fixed and lysed (15 minutes in the presence of chloroform vapour followed by 18 hours in 10OmM Tris/HCL pH 7.8; 150 mM NaCl; 5 mM MgCl2; 1.5% BSA; 1 μg/ml DNase 1; 40 μg/ml lysozyme).

Screening for colonies expressing anti-c-erbB2 reactive protein was carried out as follows:

1. Wash nitrocellulose filter three times in TNT (10 mM Tris/HCl pH 8; 150 mM NaCl; 0.05% Tween 20) then block for 60 minutes in TNT+5% dried milk protein.

2. Incubate nitrocellulose filter for 2 hours at room temperature with mouse anti-c-erbB2 antibody (Ab-3 from Oncogene Research Products, Calbiochem).

3. Wash the filter three times in TNT then incubate overnight at 4° C. with anti-mouse HRP conjugate.

4. Wash filter three times in TNT, twice in TN (10 mM Tris/HCl pH 8; 150 mM NaCl) then visualise colonies expressing anti-c-erbB2 reactive protein using chloronaphthol (6 mg chloronaphthol in TN+6 μl 30% H₂O₂).

5. After development (approximately 20 minutes treatment with chloronaphthol as described in step 4) wash filter with water and allow to air dry.

Colonies identified as positive for c-erbB2 expression were picked and grown up overnight in liquid culture of LB+ampicillin. Small amounts of plasmid DNA and protein were prepared from the culture for analysis. Plasmids containing a c-erbB2 cDNA insert were identified using restriction enzyme digestion and PCR using a primer pair specific to the published c-erbB2 cDNA sequence, described by Yazici, H. et al.(1996) Cancer Lett. 107: 235-239. DNA sequence analysis was then be used to confirm 1) the presence of a c-erbB2 insert and 2) that the reading frame of c-erbB2 is maintained in the resultant biotinylated fusion protein. Protein samples prepared from E. coli cultures carrying a plasmid with a c-erbB2 insert were analysed by SDS-PAGE and western blotting to ensure that the correct protein was being expressed.

### Example 5

### Detection of the Immune Response of Patients with Primary Breast Cancer Using a Panel Assay

Methods

(A) Preparation of Biotinylated Antigen

E. coli transformed with the appropriate PinPoint™ plasmid expressing biotinylated antigen were grown in a 5 ml overnight culture (LB+amp+biotin) and the overnight culture used to inoculate a 150 ml culture. The 150 ml culture was grown to OD 0.4-0.6 then expression of the fusion protein was induced by the addition of IPTG to a final concentration of 1 mM and the induced culture incubated at 25° C. The bacterial cells were harvested by centrifugation and then lysed by gentle sonication in a Tris/EDTA buffer containing the protease inhibitor PMSF. Cellular debris was removed by centrifugation at ˜50,000 g and the resultant particle-free supernatant assayed by avidin ELISA to confirm the presence of biotinylated protein.

(B) c-erbB2/p53 Autoantibody Assay Method

1. Standard 96 well microtitre assay plates were coated with avidin, using 50 μl of a 1 μg/ml solution per well, and allowed to air dry overnight. The plates were then washed once with PBS/Tween to remove residual salt crystals, blocked for 60 minutes with a solution of 2% (w/v) PVP (polyvinylpyrolidone 360) in PBS and washed three times using PBS/Tween.

2. Particle free supernatant containing the appropriate biotinylated antigen (prepared as described in section (1) above) was plated out at 50 μl per avidin-coated well and then incubated for 60 minutes at room temperature with shaking to allow the biotin/avidin binding reaction to take place. The plates were then washed four times with PBS/Tween.

3. Serum samples to be tested for the presence of autoantibodies (diluted 1/50 and 1/100 in PBS) were plated out in triplicate (50 μl per well) and then incubated for 60 minutes with shaking to allow formation of any autoantibody/antigen complexes. Plates were then washed four times with PBS/Tween to remove unbound serum components.

4. 50 μl of HRP conjugated anti-human IgG/IgM antibody (obtained from Dako and used at a dilution recommended by the manufacturer) was added to each well and incubated for 60 minutes at room temperature with shaking. The plates were then washed again four times with PBS/Tween.

5. 50 μl of TMB was added to each well and measurements of OD at 650 nm for each well of the assay plate were taken kinetically over a period of 10 minutes.

For each antigen, control assays were performed following the procedure described above but using a sample of protein induced from E. coli transformed with a control PinPoint™ vector containing an out-offrame cDNA instead of the particle free supernatant containing biotinylated antigen. As will be apparent to persons skilled in the art, the above methodology can be adapted for use in the detection of autoantibodies of any specificity with use of an appropriate biotinylated antigen.

(C) MUC1 Autoantibody Assay

1. ABC MUC1 isolated from the serum of patients with advanced breast cancer according to the method of Example 1 (all three fractions pooled) was diluted appropriately in PBS, plated out on a 96 well microtitre assay plate at 50 μl per well and left to dry overnight. The plate was then washed once with PBS/Tween to remove residual salt crystals, blocked for 60 minutes using a solution of 2% (w/v) PVP in PBS and washed three times with PBS/Tween.

2. Serum samples to be tested for the presence of autoantibodies (diluted 1/50 and 1/100 in PBS) were plated out in triplicate, adding 50 μl per well, and incubated for 60 minutes at room temperature with shaking. The plate was then washed four times with PBS/Tween.

3. 50 μl of HRP conjugated anti-human IgG/IgM antibody (obtained from Dako and used at a dilution recommended by the manufacturer) was added to each well and incubated for 60 minutes at room temperature with shaking. The plates were then washed again four times with PBS/Tween.

4. 50 μl of TMB was added to each well and measurements of OD at 650 nm for each well of the assay plate were taken kinetically over a period of 10 minutes.

Results

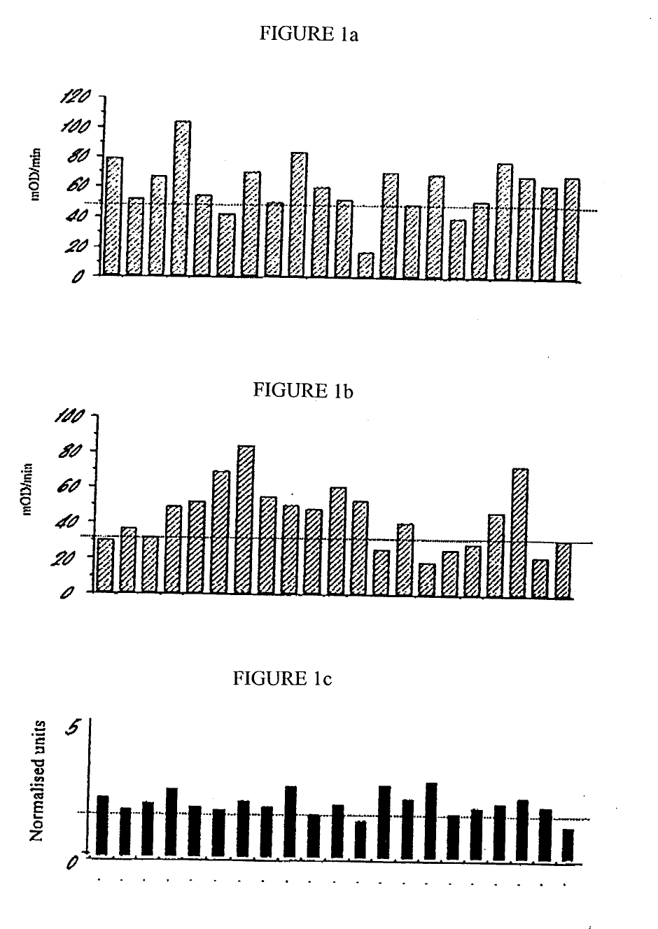

Pre-operative blood samples taken from 21 patients diagnosed with primary breast cancer were assayed for the presence of autoantibodies against MUC1, p53 and c-erbB2. The results of these assays are shown in FIG. 1 and summarised in Table 2 below.

TABLE 2

| Sam- | anti- | Pre- | anti-c- | Pre- | anti | Pre- | |
|---|---|---|---|---|---|---|---|
| ple | p53 | diction | erbB2 | diction | MUC1 | diction | Combined |
| 1 | 53 | Cancer | − | normal | + | cancer | CANCER |
| 2 | +/− | ? | +/− | ? | +/− | ? | cancer |
| 3 | + | cancer | + | ? | + | cancer | CANCER |
| 4 | + | cancer | + | cancer | + | cancer | CANCER |
| 5 | + | cancer | + | cancer | +/− | ? | CANCER |
| 6 | − | normal | + | cancer | +/− | ? | cancer |
| 7 | + | cancer | + | cancer | + | cancer | CANCER |
| 8 | +/− | ? | + | cancer | +/− | ? | CANCER |
| 9 | + | cancer | + | cancer | + | cancer | CANCER |
| 10 | + | cancer | + | cancer | − | normal | CANCER |
| 11 | +/− | ? | + | cancer | + | cancer | CANCER |
| 12 | − | normal | + | cancer | − | normal | cancer |
| 13 | + | cancer | − | normal | + | cancer | CANCER |
| 14 | +/− | ? | + | cancer | + | cancer | CANCER |
| 15 | + | cancer | − | normal | + | cancer | CANCER |
| 16 | − | normal | − | normal | +/− | ? | ? |
| 17 | +/− | ? | − | normal | + | cancer | cancer |
| 18 | + | cancer | + | cancer | + | cancer | CANCER |
| 19 | + | cancer | + | cancer | + | cancer | CANCER |
| 20 | + | cancer | − | normal | + | cancer | CANCER |
| 21 | + | cancer | +/− | ? | − | normal | cancer |

FIG. 1 shows the results of the assays for autoantibodies specific to MUC1, c-erbB2 and p53. For each set of data the dotted line represents the cut-off value for normality. For the purposes of this study the normal control patients were women who clinically and/or mammographically had no evidence of breast cancer at the time of taking the serum sample.

In order to establish the cut-off value for normality, control assays were performed on a total of 30 normal patients. Values below the dotted line fall within the normal control range and were scored as negative (−) in Table 2 whereas values above the dotted line were scored as positive (+). Values which were difficult to score as negative or positive with a reasonable degree of certainty were scored +/−. Patients scoring positive in at least two of the assays were identified as strongly positive for breast cancer (indicated “CANCER” in Table 2); patients scoring positive in at least one of the assays were identified as probable for breast cancer (indicated “cancer” in Table 2).

The results presented illustrate the predictive value of the three autoantibody assays both when used individually and when used as a panel. The use of a single assay to predict breast cancer gave approximately 40% of the results as a false negatives. However, by combining the results from all three assays only one patient appeared as a false negative (<5%), 71% of patients were scored as strongly positive for breast cancer (i.e. positive in at least two assays) and 23% of patients were scored as probable for breast cancer (i.e. positive in at least one assay). The results also show that a group of patients which have all been diagnosed with primary breast cancer have different serological profiles in terms of the immune response to their cancer. Thus, no single one of the three autoantibody assays would be useful in all primary breast cancer patients.

### Example 6

### Cloning of a ras Antigen

Method

cDNA encoding a mutant oncogenic form of ras (designated K-ras) was cloned from the cell line KNRK (Rat kidney, Kirsten MSV transformed, see Aaronson, S. A. and Weaver, C. A. (1971) J. Gen. Virol. 13: 245-252; ATCC accession number CRL 1569). mRNA was extracted from the cell pellet using a Dynabead mRNA purification kit according to the manufacturer's recommended protocol. cDNA synthesis, cloning into the EcoRV site of the PinPoint™ vector and transformation of E. coli was carried out as described in Example 4. Clones expressing ras were then identified by expression screening using the anti-ras antibody F234-4.2 from Calbiochem.

### Example 7

### Cloning of c-myc

Method

cDNA encoding human c-myc was cloned from the breast cancer cell line T47-D (European Collection of Animal Cell Cultures accession number 85102201). mRNA was extracted from the cell pellet using a Dynabead mRNA purification kit according to the manufacturer's recommended protocol. cDNA synthesis, cloning into the EcoRV site of the PinPoint™ vector and transformation of E. coli was carried out as described in Example 4. Clones expressing c-myc were then identified by expression screening using the anti-c-myc antibody 4111.1 from Unilever.

### Example 8

### Assay for ras and c-myc Autoantibodies

Biotinylated c-myc and ras antigens were prepared from E. coli transformed with the appropriate PinPoint™ plasmid vector expressing biotinylated c-myc or biotinylated ras, as described in Example (5), part (A). The assays for c-myc and ras autoantibodies were then performed according to the protocol described in Example (5), part (B).

### Example 9

### Method of Detecting Recurrent Disease in a Patient Previously Diagnosed as Carrying Tumour Cells

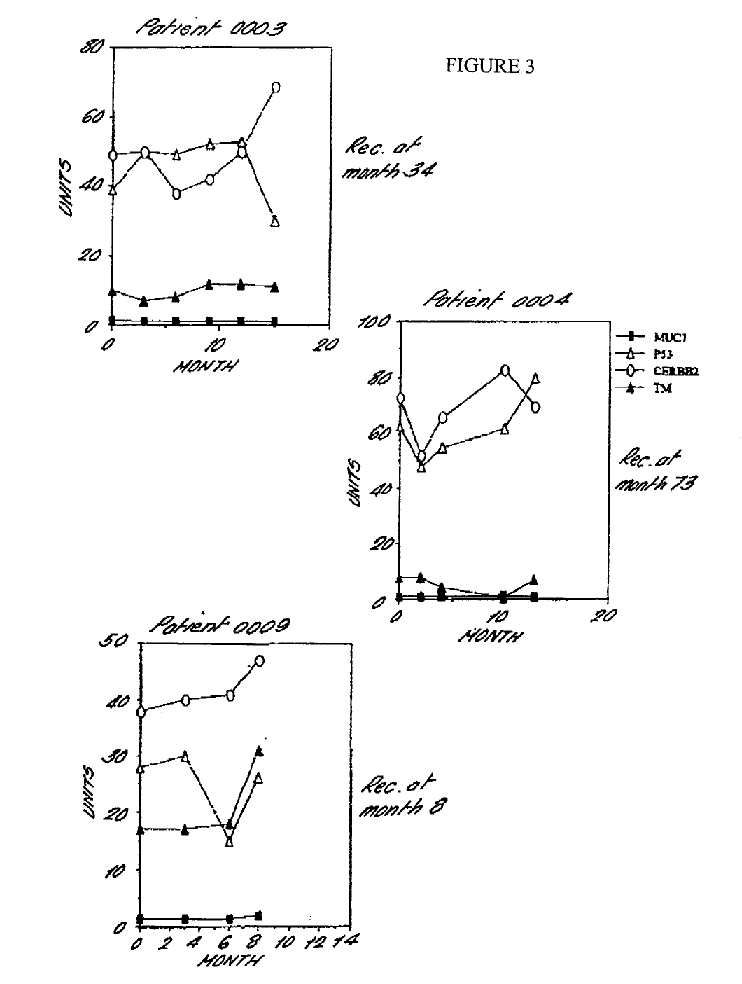

A group of nine patients previously diagnosed with primary breast cancer were selected. Pre-operative serum samples were taken from each of these patients prior to surgery for the removal of the primary breast cancer. Follow-up serum samples were then taken post-operatively at 2 or 3 monthly intervals and during the same period of time the patients were assessed clinically for signs of recurrent disease. None of the patients received any post-operative therapy until recurrence was diagnosed clinically. The pre-operative and post-operative serum samples from each of the patients were assayed for the presence of autoantibodies to MUC1, c-erbB2 and p53, using the assay methods described above under Example 5, and also for the presence of the commonly used serum tumour marker protein CA15-3. The results of these assays are summarised in Table 3, on pages 35 and 36 and results for three of the nine patients are presented graphically in FIG. 3.

Clinical signs of recurrent disease were scored as follows:

| | |
|---|---|
| LN | recurrent disease in the lymph nodes |
| LR | local recurrence |
| METS | distant metastases present |

Results

In each of the patients at least one class of autoantibody was observed to remain above normal level. This suggests continued presence of the tumour marker (immunogen) and hence continued presence of tumour. Serum levels of the tumour marker protein CA15-3 were not found to be predictive of recurrent disease.

TABLE 3

| | | | Anti- | Pre- | Anti-c- | Pre- | Anti- | Pre- | Pre- | Re- | Date of 1st | DFI |
|---|---|---|---|---|---|---|---|---|---|---|---|---|
| Patient | Sample Date | CA15-3 | p53 | diction | erb B2 | diction | MUC 1 | diction | dicted | currence | Recurrence | (Months) |
| 0001 | December 1988 | 11 | − | | + | cancer | + | cancer | CANCER | − | | |
| | March 1987 | 12 | − | | + | cancer | + | cancer | CANCER | − | | |
| | May 1987 | 13 | − | | + | cancer | + | cancer | CANCER | − | | |
| | August 1987 | 22 | +/− | ? | + | cancer | + | cancer | CANCER | − | | |
| | November 1987 | 56 | +/− | ? | + | cancer | + | cancer | CANCER | METS | | |
| | December 1987 | 79 | +/− | ? | + | cancer | + | cancer | CANCER | METS | | 11 |
| 0002 | January 1987 | 16 | − | | + | cancer | +/− | ? | Cancer | − | | |
| | May 1987 | 8 | − | | + | cancer | +/− | ? | Cancer | − | | |
| | August 1987 | 10 | − | | + | cancer | + | cancer | CANCER | − | | |
| | November 1987 | 12 | +/− | ? | + | cancer | + | cancer | CANCER | − | | |
| | February 1988 | 16 | − | | + | cancer | + | cancer | CANCER | − | February 1989 | 23 |
| 0003 | February 1987 | 10 | − | | + | cancer | − | | Cancer | − | | |
| | May 1987 | 7 | + | cancer | + | cancer | − | | CANCER | − | | |
| | August 1987 | 8 | + | cancer | + | cancer | − | | CANCER | − | | |
| | November 1987 | 12 | + | cancer | + | cancer | − | | CANCER | − | | |
| | February 1988 | 12 | + | cancer | + | cancer | − | | CANCER | − | | |
| | May 1988 | 11 | − | | + | cancer | − | | Cancer | − | December 1989 | 34 |
| 0004 | February 1987 | 8 | + | cancer | ++ | cancer | − | | CANCER | − | | |
| | April 1987 | | + | cancer | + | cancer | − | | CANCER | − | | |
| | June 1987 | 4 | + | cancer | + | cancer | − | | CANCER | − | | |
| | December 1987 | 0.4 | + | cancer | ++ | cancer | − | | CANCER | − | | |
| | March 1988 | 7 | ++ | cancer | ++ | cancer | − | | CANCER | − | February 1993 | 71 |
| 0005 | March 1987 | 16 | +/− | ? | + | cancer | − | | Cancer | − | | |
| | June 1987 | 13 | +/− | ? | + | cancer | − | | Cancer | | | |
| | September 1987 | 14 | + | cancer | + | cancer | +/− | ? | CANCER | | | |
| | December 1987 | 17 | +/− | ? | + | cancer | +/− | ? | CANCER | | | |
| | March 1988 | 16 | | | | | | | | − | | |
| | May 1988 | | | | | | | | | LN | | 15 |
| 0006 | May 1987 | 12 | − | | + | cancer | + | cancer | CANCER | − | | |
| | July 1987 | 15 | − | | + | cancer | + | cancer | CANCER | − | | |
| | September 1987 | 9 | +/− | ? | + | cancer | +/− | ? | Cancer | LR | | 4 |
| | November 1987 | 12 | − | | + | cancer | +/− | ? | Cancer | − | | |
| | March 1988 | 15 | − | | +/− | ? | − | | | − | | |
| | May 1988 | 13 | − | | +/− | ? | − | | | − | | |
| 0007 | June 1987 | 26 | + | cancer | ++ | cancer | − | | CANCER | − | November 1988 | |
| | August 1987 | 28 | + | cancer | + | cancer | − | | CANCER | − | | |
| | October 1987 | 42 | + | cancer | + | cancer | − | | CANCER | − | | |
| | December 1987 | 105 | + | cancer | ++ | cancer | + | cancer | CANCER | METS | December 1987 | 6 |
| 0008 | June 1987 | 48 | + | cancer | + | cancer | + | cancer | CANCER | − | | |
| | August 1987 | 30 | + | cancer | + | cancer | + | cancer | CANCER | − | | |
| | October 1987 | 17 | + | cancer | + | cancer | + | cancer | CANCER | − | | |
| | January 1988 | 14 | + | cancer | + | cancer | + | cancer | CANCER | − | | |
| | May 1988 | 22 | + | cancer | + | cancer | +/− | ? | CANCER | LR | May 1988 | 11 |
| 0009 | May 1987 | 17 | − | | +/− | ? | − | | | − | | |
| | August 1987 | 17 | − | | + | cancer | − | | Cancer | − | | |
| | November 1987 | 18 | − | | + | cancer | − | | Cancer | LR | | 6 |
| | January 1988 | 31 | − | | + | cancer | +/− | ? | Cancer | METS | | 8 |

### Example 10

### Retrospective Analysis of a Well Characterised Series of Healthy Controls and Patients with Early Breast Cancer

The above-described methods for detecting autoantibodies to MUC1, p53, c-erbB2 and c-myc were used to carry out a retrospective study on a large number of early (stage 1 and 2) breast cancer sera as well as a large number of control serum samples from individuals with no evidence of malignancy (control group). The serum samples from patients were all taken within a 4 week pre-operative period. At the same time, the serum samples were assayed for the presence of circulating antigen (MUC1 and c-erbB2) using conventional tumour marker kits (used normally in advanced disease only). This allowed an assessment of whether the autoantibody assays are more sensitive than the conventional antigen assays. As used herein, the terms early or primary breast cancer means that the primary tumour has a diameter of less than 5 cm. Stage 1 early breast cancer is defined as lymph node negative; Stage 2 early breast cancer is defined as lymph node positive.

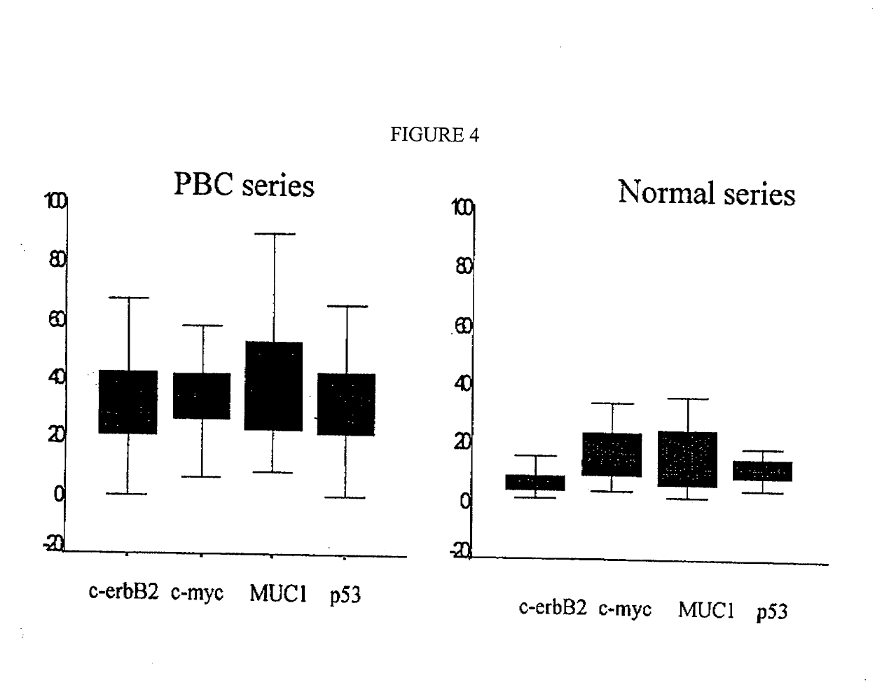

In total, pre-operative serum samples from 200 patients diagnosed with primary breast cancer and 100 normal control samples were assayed for autoantibodies against MUC1, p53, c-erbB2 and c-myc. The results are summarised in Tables 4-7 and FIGS. 4-7.

FIG. 4 depicts the range of autoantibody levels found for each assay in normal individuals and patients with early breast cancer. It is apparent that cancer patients have a considerably higher level of circulating autoantibodies to these markers than do normal individuals. Using the range for the normal individuals it is possible to set a ‘cut-off’ above which no normal values should lie. Therefore, samples with autoantibody levels above this cut-off can be deemed to be positive for cancer. Cut-off points determined in this manner were used to score the results of the retrospective study in early breast cancer patients.

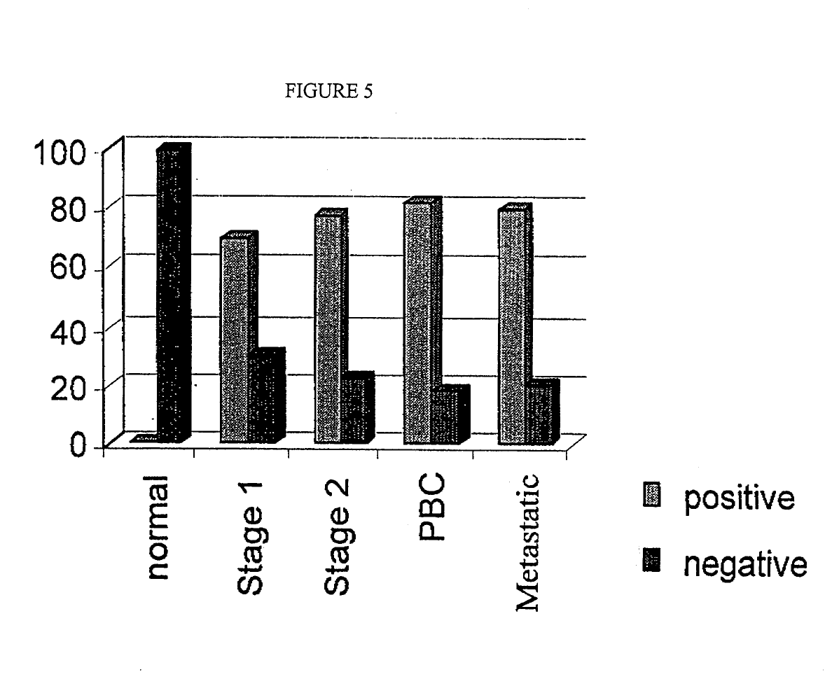

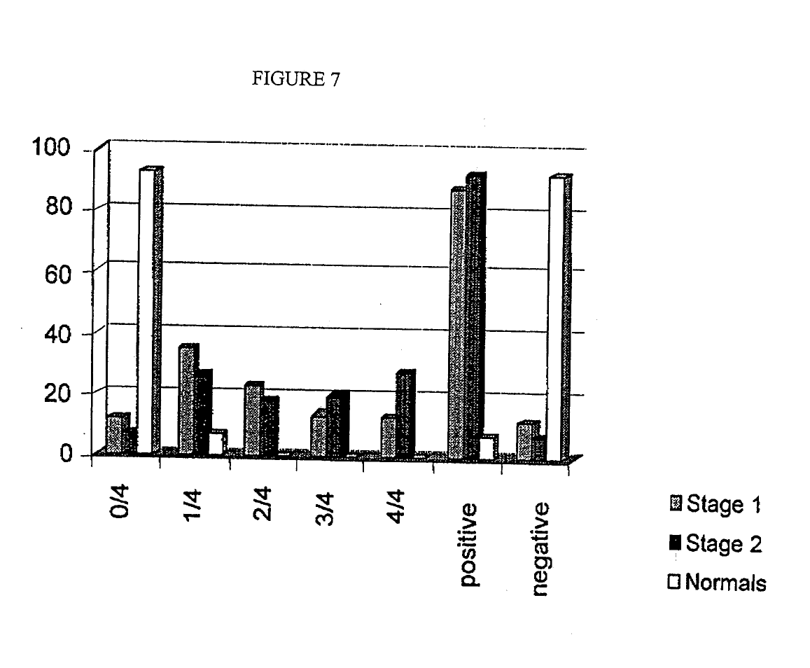

The results presented in Tables 4-7 and FIGS. 5-7 demonstrate the predictive value of the four autoantibody assays both individually and when used in combination as a panel of assays. Table 4 indicates the increased sensitivity of combining the results of a number of assays. By using one assay on its own, less than 50% of cancers are detected, however the power of detection increases as more assays are added to the panel until the combination of all four assays allows 82% of primary cancers to be detected. FIG. 7 shows the percentage of samples which are positive in 0 out of 4 assays up to 4 out of 4 assays. This provides good evidence that the panel assay is more powerful in the detection of cancer than any one single marker assay since not all patients with cancer have raised autoantibodies to all markers.

Tables 5-7 summarise the detection rates in stage 1, stage 2 and in early breast cancer (i.e. stage 1 and 2) for various combinations of autoantibody assays. The use of a single autoantibody assay to predict breast cancer gives approximately 60-70% of the results as false negatives in the stage 1 group; and 50-60% in stage 2. However, by combining the results from all four assays, 76% of stage 1 and 89% of stage 2 cancers were positive in one or more assay. The overall detection rate for early breast cancer (i.e. both stage 1 and stage 2 cancers) using this system was 82%. In both stage 1 and stage 2 cancer, assaying for autoantibodies to MUC1 appeared to add predictive power to any combination of assays.

The results for this study were obtained using a 100% confidence limit, in other words for a result to be deemed positive it had to fall above the cut-off for readings in the normal range. This normal range was previously evaluated from a large number of normal individuals and then confirmed using the control group of 100 normal individuals mentioned above. Therefore, within the normal control group, none of the samples were found to be positive, meaning that the sensitivity of the panel of autoantibody assays was 100% for the detection of early breast cancer (FIG. 5).

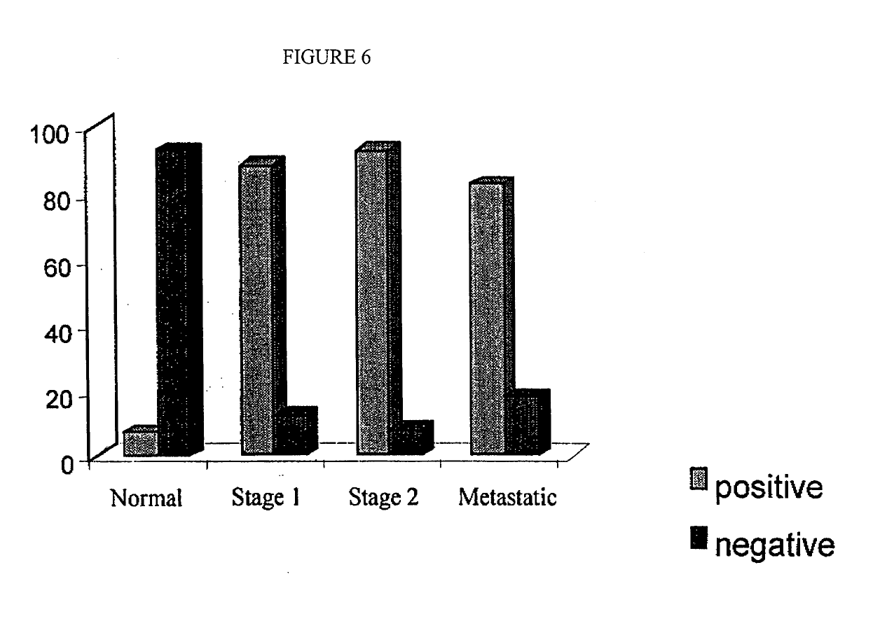

FIGS. 6 and 7 demonstrate the detection rates which are achievable if specificity is reduced from a 100% confidence level (no false positives) to a 95% confidence level, where some degree of false positive detection is expected. In this case, the cut-off point is defined as the mean value plus twice the standard deviation of the normal sample range. Using this cut-off point, approximately 5% of the normal samples were determined to be positive for cancer (i.e. false positives); whilst detection of primary cancer increased to approximately 94% (i.e. 6% false negatives). Again, the greatest percentage of the sample group were positive in only 1 out of the 4 assays, however, the percentage of samples that were positive in all 4 assays increased considerably.

Since the above study was carried out retrospectively, clinical data was available regarding the initial diagnosis as well as clinical data regarding the post-operative outcome (i.e. follow-up data). This allowed analysis of the prognostic value of the data obtained from the autoantibody assays. Table 8 shows the correlations between serum levels of autoantibodies to MUC1, p53, c-erbB2 and c-myc and a number of clinical factors. For instance, the presence of autoantibodies to any of the 4 tumour associated proteins (MUC1, p53, c-erbB2 or c-myc) appears to correlate with the development of a recurrence. In other words, those patients who had autoantibodies were more likely to go on to develop a recurrence of their disease. In the case of autoantibodies to MUC1, c-myc and c-erbB2, this was most likely to be distant metastases, only autoantibodies to p53 were not associated with the later development of distant metastases with any statistical significance. In fact, the presence of autoantibodies to p53 was the weakest indicator of a later recurrence of disease; furthermore, p53 autoantibodies correlated with disease free interval.

Table 9 presents an analysis of whether the degree of autoantibody positivity may be of value in the prediction of which stage 1 tumour will go on to develop a recurrence. At the present time, there is little to indicate at the time of diagnosis whether a patient with a stage 1 tumour (i.e. no evidence of spread of tumour to the lymphatic system) will go on to develop recurrent disease. As can be seen in Table 9, of those patients with stage 1 tumours from the sample group that went on to develop recurrent disease, 71% were positive in two or more autoantibody assays. Of the patients with stage 1 tumours that have not yet recurred, only 30% were positive in two or more autoantibody assays.

TABLE 4
Sensitivity of autoantibody assays in
the detection of early breast cancer.

| | % PBC positive |
|---|---|
| Single marker assay | 35-47 |
| Two marker assay | 51-60 |
| Three marker assay | 63-76 |
| Four marker assay | 82 |

TABLE 5
Sensitivity of autoantibody panel assays
in the detection of stage 1 breast cancer.

| | p53 | c-erbB2 | c-myc | MUC1 |
|---|---|---|---|---|
| p53 | 38 | 48 | 58 | 59 |
| c-erbB2 | | 31 | 50 | 51 |
| c-myc | | | 41 | 55 |
| MUC1 | | | | 38 |
| p53/c-erbB2 | | | 61 | 66 |
| p53/c-myc | | | | 73 |
| c-erbB2/c-myc | | | | 65 |
| p53/c-erbB2/c-myc | | | | 76 |

TABLE 6
Sensitivity of autoantibody panel assays
in the detection of stage 2 breast cancer.

| | p53 | c-erbB2 | c-myc | MUC1 |
|---|---|---|---|---|
| p53 | 40 | 56 | 55 | 73 |
| c-erbB2 | | 42 | 56 | 73 |
| c-myc | | | 33 | 69 |
| MUC1 | | | | 56 |
| p53/c-erbB2 | | | 65 | 84 |
| p53/c-myc | | | | 80 |
| c-erbB2/c-myc | | | | 84 |
| p53/c-erbB2/c-myc | | | | 89 |

TABLE 7
Sensitivity of autoantibody panel assays
in the detection of primary breast cancer.

| | p53 | c-erbB2 | c-myc | MUC1 |
|---|---|---|---|---|
| p53 | 38 | 16 | 57 | 64 |
| c-erbB2 | | 35 | 53 | 59 |
| c-myc | | | 37 | 60 |
| MUC1 | | | | 47 |
| p53/c-erbB2 | | | 63 | 73 |
| p53/c-myc | | | | 76 |
| c-erbB2/c-myc | | | | 72 |
| p53/c-erbB2/c-myc | | | | 82 |

TABLE 8
Correlations between serum autoantibody
level and various clinical factors.

| FACTOR | MUC1 | p53 | c-erbB2 | c-myc |
|---|---|---|---|---|
| recurrence | * | 1*4 | * | * |
| local recurrence | 1*2 | 1*2 | 1*2 | 1*4 |
| distant metastases | * | + | * | * |
| stage | + | + | + | + |
| grade | + | + | + | + |
| family history | + | + | + | + |
| disease free interval | + | + | + | + |
| age | + | + | + | + |
| menopausal status | + | + | + | + |

Key:
*Good correlation
1*2 Moderate correlation
1*4 Weak correlation
+ No correlation

TABLE 9
Analysis of the degree of positivity in autoantibody assays
for recurrent and non-recurrent stage 1 breast cancer tumours.

| | Negative—no | +ve auto- | +ve auto- |
|---|---|---|---|
| | autoantibodies | antibodies to | antibodies to |
| | detected | one marker | 2-4 markers |
| Recurrent | 12% | 17% | 71% |
| Non-recurrent | 22% | 48% | 30% |

### Example 11

### Detection of Autoantibodies in Sequential Serum Samples-Application to the Monitoring of Disease Progression

This study was carried out in order to assess whether autoantibody assays are useful in the earlier detection of recurrent disease.

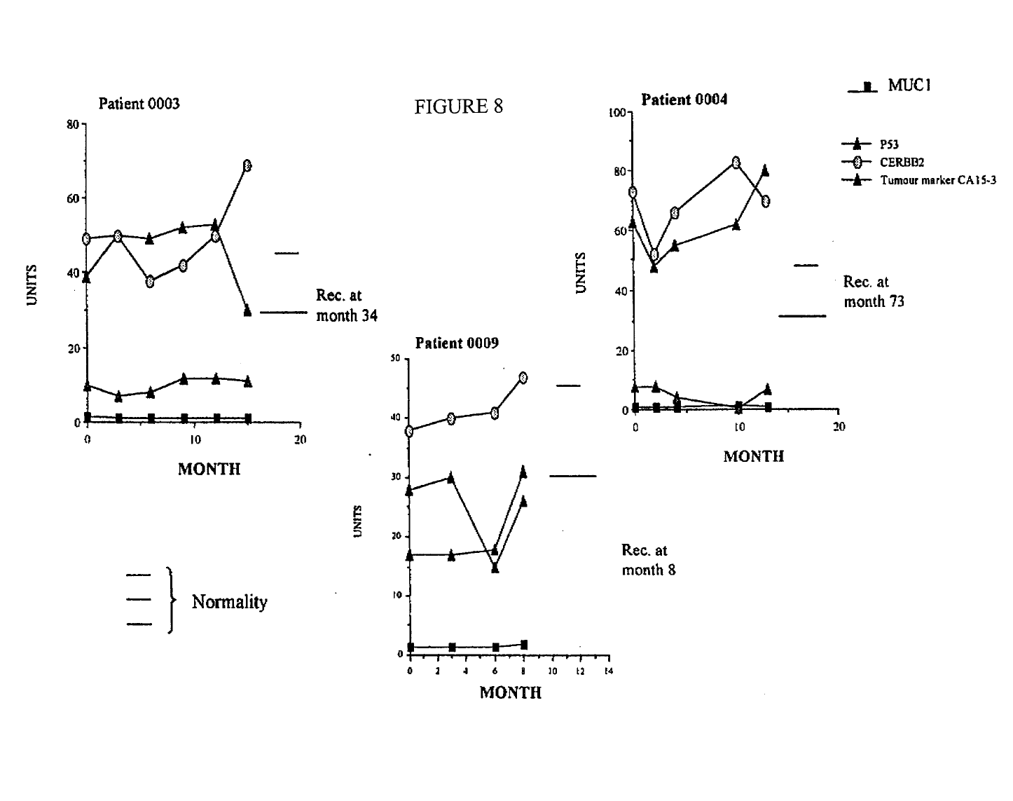

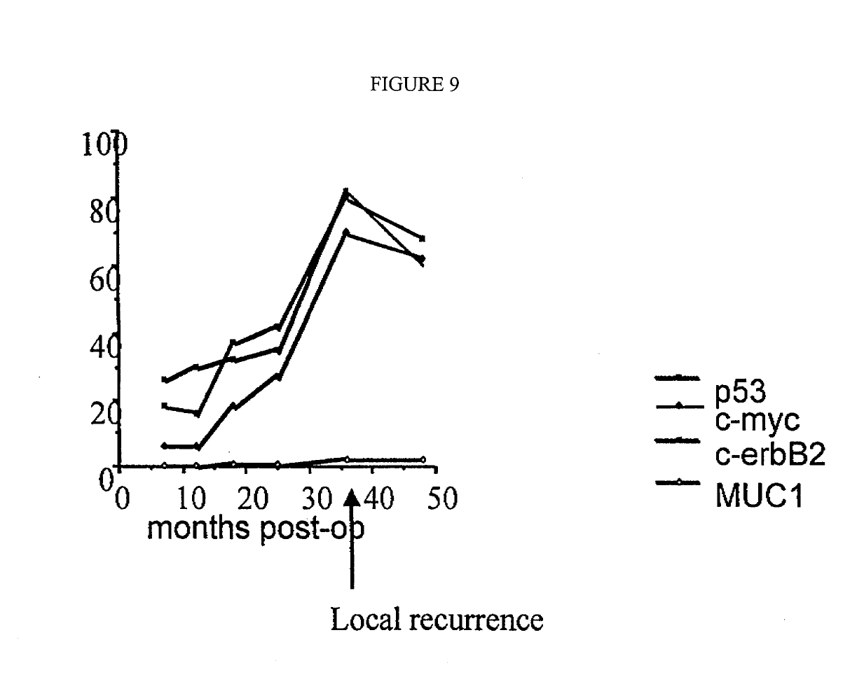

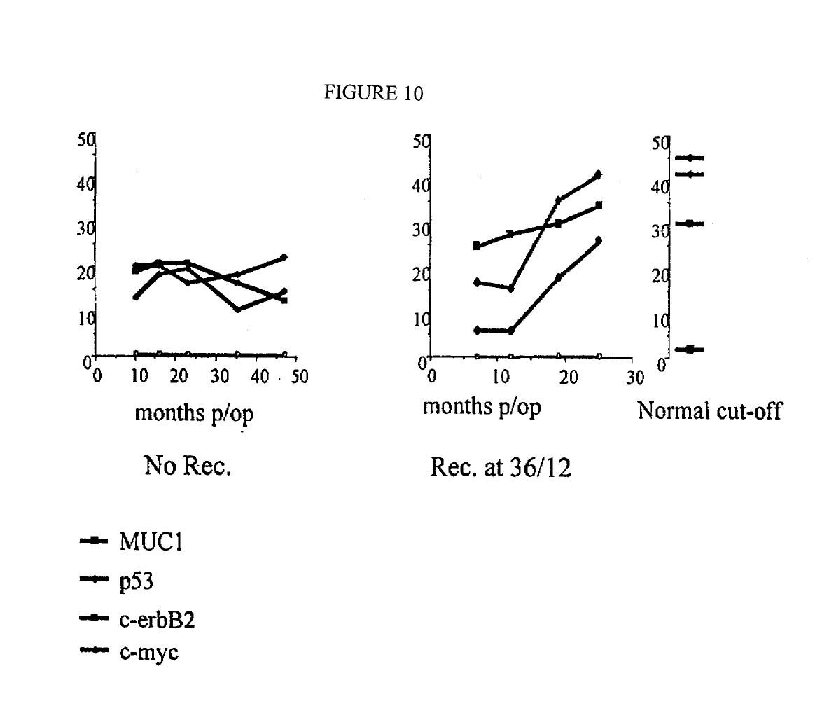

Levels of autoantibodies to MUC1, p53 and c-erbB2 in the serum of patients previously diagnosed with breast cancer were measured sequentially during follow-up until the patient manifested recurrent disease. The results are summarised in FIGS. 8-10. All three patients went on to develop recurrent disease. In all three patients, autoantibody levels were indicative of the presence of cancer. However, there is no evidence from this group that autoantibody levels decrease after removal of the primary tumour. FIG. 10 shows the levels of autoantibodies post-operatively of a patient with non-recurrent disease and a patient with recurrent disease. Autoantibody levels in the patient with non-recurrent disease remained below the cut-off point during the period of sample collection (48 months). In the second patient, whose disease recurred at 36 months, autoantibody levels are seen to be steadily rising towards the cut-off point, with c-erbB2 autoantibodies rising above cut-off. Furthermore, as can be seen in FIG. 9, when further sequential samples are added to the analysis, 3 out of the 4 assays become positive for cancer and these levels then decrease again once treatment of the recurrence is underway. Sequential measurements of established tumour markers reflecting tumour bulk (e.g. CA15-3 and CEA) were within the normal range throughout this period (data not shown). This data supports the utility of autoantibody assays in the earlier detection of recurrent disease.

### Example 12

### Analysis of a Series of Patients with Bladder Cancer and Benign Urological Disorders

Serum samples were collected from a group of 80 patients with bladder cancer/benign urological disorders and analysed for the presence of autoantibodies to MUC1, p53, c-erbB2 and c-myc using the assay methods described above.

The data summarised in Table 10 shows that single assay sensitivities for bladder cancer detection range from 15-50% (as opposed to 35-47% for breast cancer). The detection sensitivity using all 4 assays was 80%, similar to that found for early breast cancer.

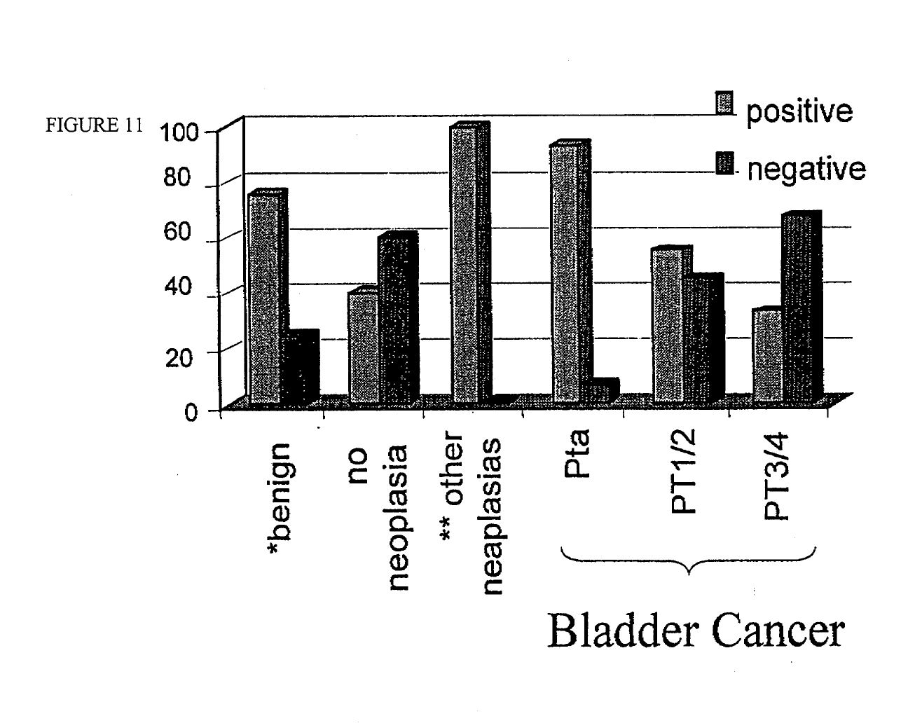

FIG. 11 shows the break down of detection rates between urologically benign disorders (‘benign’) and the three stages of bladder cancer. Upon further investigation of the relevant clinical data it became apparent that 6 of the patients in the ‘benign’ group had evidence of other malignancies. The * indicates patients which were benign with respect to urology (i.e. did not have a urological malignancy). The ** indicates the six cases (all with positive autoantibody status) which had evidence of lung cancer, skin cancer, adenocarcinoma of unknown primary. Evidence of other neoplasia consisted of:—pleural effusion, ovarian cysts, colon polyps. Serum samples from all 6 of these patients had been scored as positive for cancer using the panel of autoantibody assays, illustrating the general application of the panel assay to the detection of cancers. Furthermore, it is known that some patients with stage PT1/2 and PT3/4 disease had previously received systemic therapy.

TABLE 10
Sensitivity of autoantibody assays
in the detection of bladder cancer.

| | % positive |
|---|---|
| Single marker assay | 15-50 |
| Two marker assay | 28-73 |
| Three marker assay | 46-76 |
| Four marker assay | 80 |

TABLE 11
Sensitivity of autoantibody panel assays
in the detection of bladder cancer.

| | p53 | c-erbB2 | c-myc | MUC1 |
|---|---|---|---|---|
| p53 | 50 | 73 | 73 | 73 |
| c-erbB2 | | 17 | 28 | 36 |
| c-myc | | | 15 | 35 |
| MUC1 | | | | 24 |
| p53/c-erbB2 | | | 76 | 76 |
| p53/c-myc | | | | 75 |
| c-erbB2/c-myc | | | | 46 |
| p53/c-erbB2/c-myc | | | | 80 |

### Example 13

### Sensitivity of Autoantibody Assay in Diagnosis of Colorectal Cancer

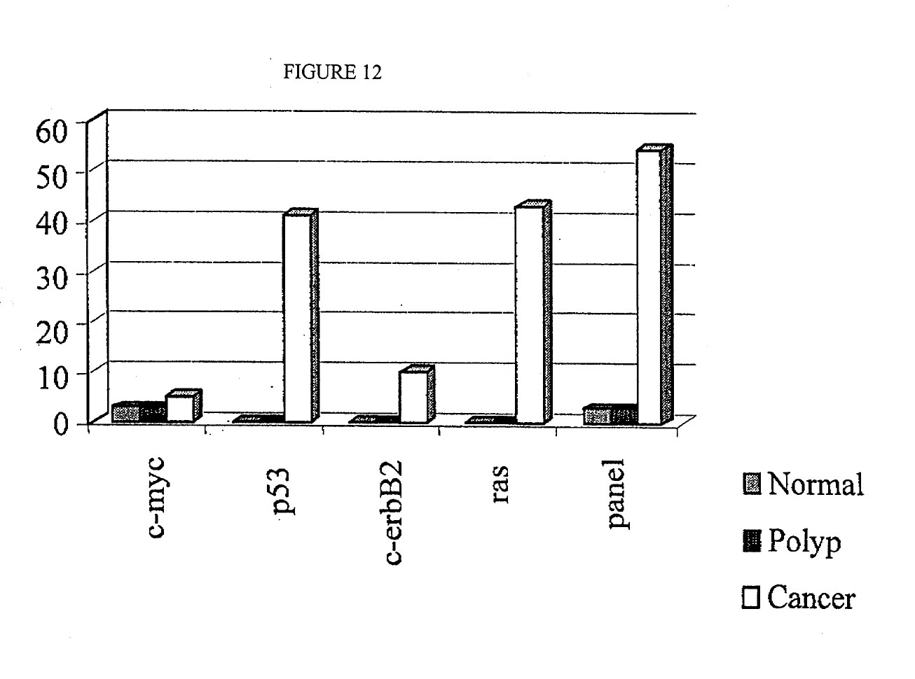

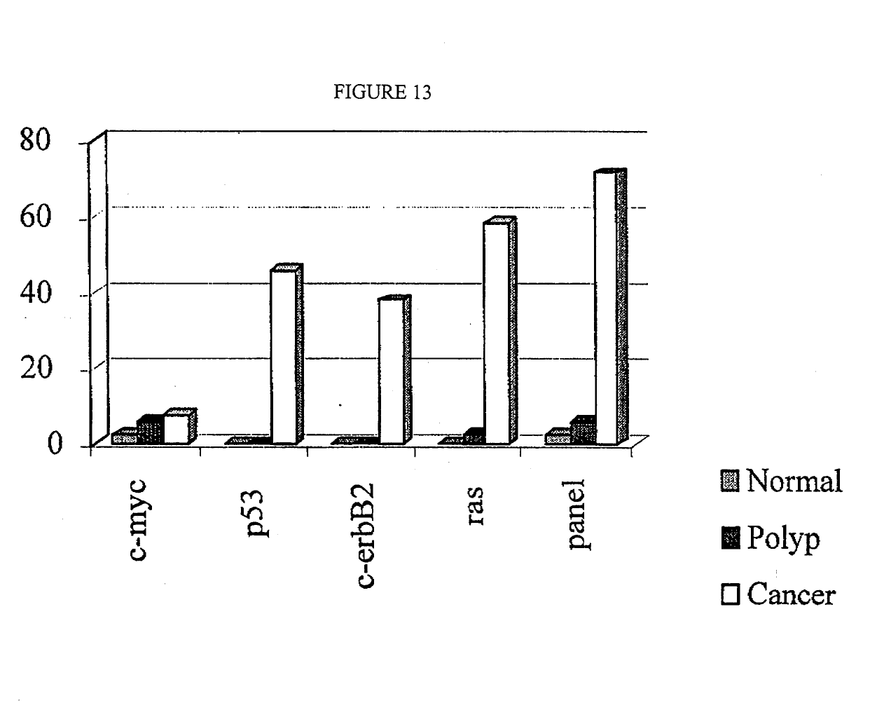

An autoantibody assay as previously described was carried out on serum samples from patients with colorectal cancer using the tumour antigens c-myc, p53, c-erbB2 and K-ras individually and as a panel. The results are shown in FIGS. 12 and 13. As has been demonstrated previously increased sensitivity is shown when a panel of antigens is used.

### Example 14

### Use of BRCA1 in Panel Assay for Detection of Breast Cancer

A BRCA1 antigen suitable for use in the detection of anti-BRCA1 autoantibodies was cloned from the breast cancer cell line MCF7 using an RT-PCR strategy. Briefly, mRNA isolated from MCF7 cells was reverse transcribed to give first-strand cDNA. These cDNA was used as a template for PCR using a primer pair designed to amplify a product covering the first 1500 base pairs of the BRCA1 cDNA but including a known mis-match mutation that leads to an early stop codon and therefore the production of truncated protein. Different sites for restriction enzyme digestion were also incorporated into the forward and reverse PCR primers to facilitate the cloning of the PCR product. The PCR primers were as follows:

| | |
|---|---|
| 5′-GAC AGG ATC CGG ATG GAT TTA TCT GCT CTT CGC GTT G | (SEQ ID NO:1) |
| 5′-GCG GCC GCC CTC ATG TAG GTC TCC TTT TAC GC | (SEQ ID NO:2) |

The PCR product obtained using these primers was then cloned into the PinPoint™ vector and used to transform E. coli Top 10 F cells, as described hereinbefore. Clones expressing the fusion protein of truncated BRCA1 antigen fused in-frame to the N-terminal biotinylation domain were then identified by expression screening, according to the procedure described in Example 4, using the antibody MAB4132 from Chemicon.

Biotinylated truncated BRCA1 antigen is then prepared from E. coli transformed with the appropriate PinPoint™ plasmid vector expressing the fusion protein, as described in Example (5), part (A). The assay for BRCA1 auto antibodies is then performed according to the protocol described in Example (5), part (B).

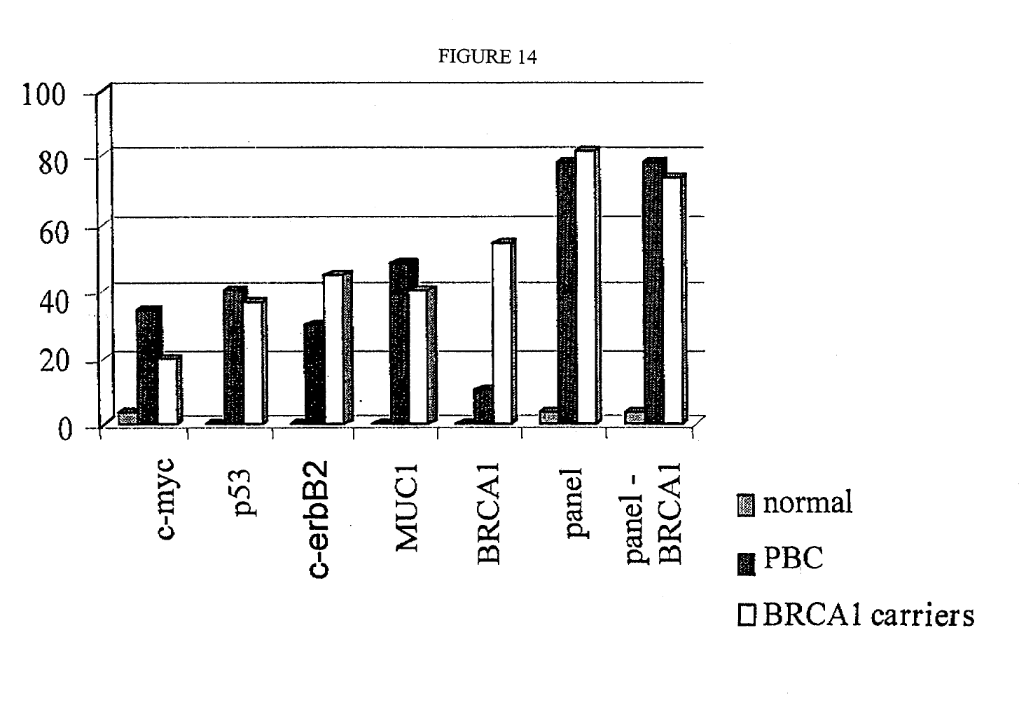

FIG. 14 shows the results of a study in which the above-described assays for autoantibodies to c-myc, p53, c-erbB2, MUC1 and BRCA1 were performed individually, as a panel and as a panel without BRCA1 to detect autoantibodies in samples of serum taken from normal individuals, patients diagnosed with primary breast cancer and BRCA1 mutation carriers. As demonstrated previously, increased sensitivity is shown when a panel of markers is used.

### Example 15

### Use of Auto Antibody Panel Assay for Detecting Prostate Cancer, Incorporating PSA

cDNA encoding human PSA was cloned from the cell line T47-D using a protocol similar to that described above for the cloning of c-erbB2. Briefly, the T47-D cells were first stimulated with Apigenin at 10-5M as described by Rosenberg et al. (1998) Biochem Biophys Res Commun. 248: 935-939. mRNA was then extracted and cDNA synthesis, ligation into PinPoint™ and transformation of E. coli. performed as described in Example 4. Clones expressing PSA were identified using an anti-PSA antibody. Biotinylated PSA antigen was prepared from E. coli transformed with the PinPoint™ vector expressing biotinylated PSA according to the protocol described in Example (5), part (A). The assay for PSA autoantibodies was then performed according to the protocol described in Example (5), part (B).

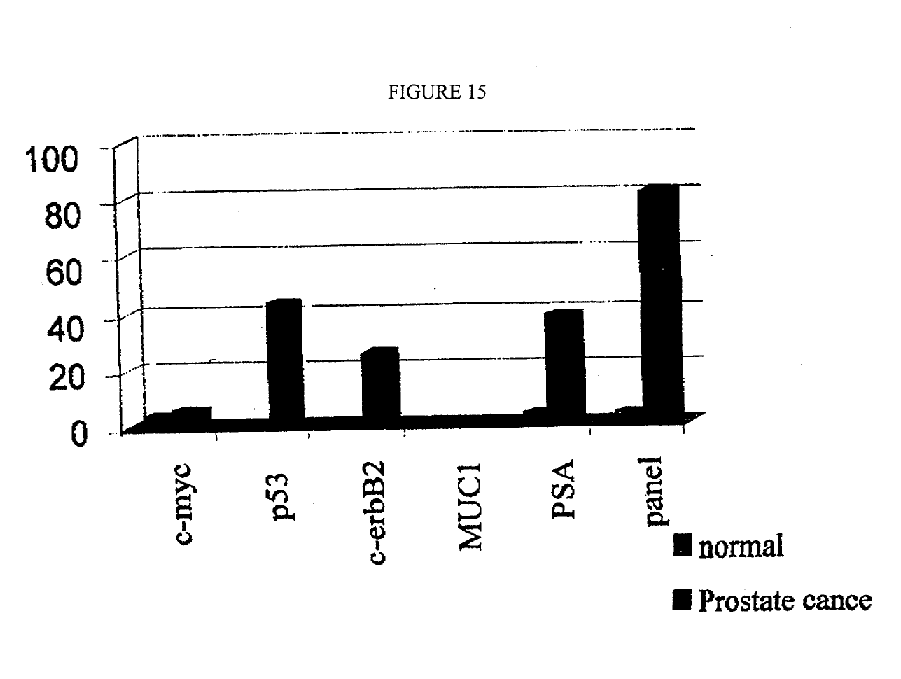

An autoantibody assay using the methods described above was carried out on patients with prostate cancer using c-myc, p53, c-erbB2, PSA and MUC1 individually and as a panel. The results are shown in FIG. 15 and confirm the increased sensitivity of such a panel for detection of prostate cancer.

### Example 16

### Other Tumour Marker Antigens

CA125 can be affinity purified from the ovarian cancer cell line OVRCAR-3 (available from the ATCC) using Mab VK-8, as described by Lloyd, K. O. et al. (1997) Int. J. Cancer. 71: 842-850.

APC protein is expressed by the colorectal cancer cell line SW480 (available from the ATCC) as described by Munemitsu, S. et al. (1995) PNAS 92: 3046-3050.

### Example 17

### Immortalisation of Mononucleocytes

Peripheral blood mononucleocytes were purified from a 4 ml sample of heparinised blood from patients or normal individuals using lymphocyte separation medium (ICN flow), as described in detail in the manufacturers instructions. Isolated mononucleocytes were washed in PBS and resuspended in 1 ml of a semipurified preparation of Epstein Barr Virus (EBV) from the B95-8 marmoset transformed leukocyte EBV-producing cell line. The cells were then incubated for 1 hour at 37° C. in 5% CO₂ and centrifuged at 17000 rpm. The EBV supernatant was removed and the mononucleocytes were washed three times with RPMI medium, resuspended in RPMI medium supplemented with 10% fetal bovine serum and 5 μg/ml phytoheamatagglutinin (PHA-P) and seeded in multi-wells tissue culture plates. The medium was changed every 3 days and used as a source of autoantibodies. B cells immortalized in the way are known to secrete their antibody for a period of up to 2 weeks with maximum secretion of the antibody into the culture medium occurring around day 10.

### Example 18

### Assessment of the Reactivity of Autoantibodies with MUC1 Antigen from Different Sources

Methods:

1) Immunoaffinity Purification of MUC1 Antigen

MUC1 was purified from the serum of patients diagnosed with primary breast cancer or advanced breast cancer or from the urine of healthy subjects according to the following protocol.

The mouse monoclonal B55 antibody (also known as NCRC 11 as described by Ellis et al. (1984) Histopathology 8: 501-516 and in International Patent Application No. WO 89/01153) was conjugated to CNBr sepharose beads. Serum or urine samples were diluted 1/10 in PBS and incubated with the antibody conjugated sepharose beads overnight at 4° C. with rolling. The beads were centrifuged and the supernatant removed. In order to remove any molecule non-specifically bound to the beads, these were washed in PBS for 5 times or until the washing buffer showed no absorbance at 280 nm. Each wash was performed by resuspending the beads in PBS, rolling for 10 minutes, centrifuging and removing the supernatant. The washed beads were resuspended in 0.25 M glycine pH 2.5, rolled at room temperature for 10 minutes and centrifuged. The supernatant was removed, adjusted to pH 7 by addition of TRIS and stored at 4° C. labelled “glycine fraction”. The beads were then resuspended in 25 mM diethylamine (DEA) pH 11, rolled at room temperature for 10 minutes and centrifuged. The supernatant was again removed, adjusted to pH 7 by addition of TRIS and stored at 4° C. labelled “25 DEA fraction”. The beads were finally resuspended in 100 mM DEA pH 11, rolled at room temperature for 10 minutes and centrifuged. The supernatant was removed, adjusted to pH 7 by addition of TRIS and stored at 4° C. labelled “100 DEA fraction”. The presence of MUC1 in the three fractions were confirmed by ELISA using the monoclonal antibody B55 or C595 (also known as NCRC, available from the Cancer Research Campaign). In order to remove contaminating immunoglobulins, fractions were incubated with DTT (to 50 mM) for 30 minutes, then iodacetamide (to 75 mM) before being subjected to gel filtration on a S300 column. Fractions were assayed for MUC1 content by ELISA. MUC1 containing fractions are titrated so as to give equivalent absorbances to previous batches.

2) ELISA Assay

Different MUC1 preparations, obtained as described above, were appropriately diluted with PBS and plated out at 50 μl per well in a 96 well microtitre assay plate and left to dry overnight. The plate was then washed once with PBS/Tween to remove residual salt crystals, blocked for 60 minutes with a fresh solution of 2% (w/v) polyvinylpyrrolidone (PVP) in PBS and washed three times with PBS/Tween. Culture supernatant of immortalised lymphocytes derived from patients diagnosed with primary or secondary breast cancer were plated out in triplicate, at 50 μl per well. As a comparative control the mouse monoclonal anti-MUC1 antibody B55 was also plated in triplicate. The plate was incubated for 60 minutes at room temperature with shaking and washed four times with PNS/Tween. 50 μl of HRP conjugated anti-human or anti-mouse secondary antibody (obtained from Dako) were added to each well at the dilution recommended by the manufacturer, and incubated for 60 minutes at room temperature with shaking. The plate was then washed again four times with PBS/Tween. 50 μl of TetraMethylBenzidine (TMB) were added to each well and optical density (OD) at 650 nm for each well of the assay plate was read kinetically over a period of 10 minutes.

Results:

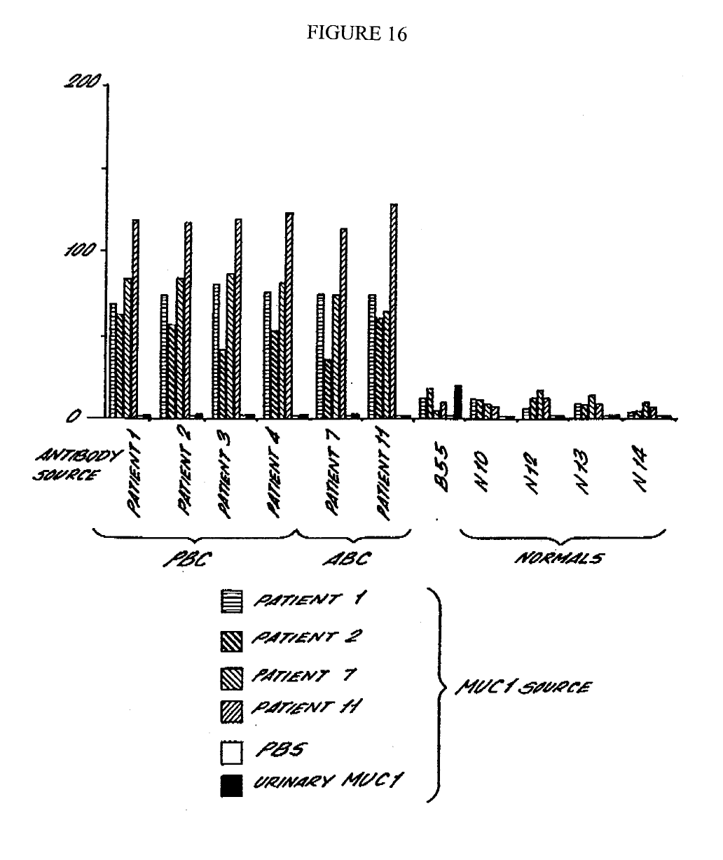

FIG. 16 shows the result of an ELISA assay to assess the reactivity of autoantibodies produced by lymphocytes derived from six patients diagnosed with breast cancer (1 to 4, with primary breast cancer, 7 and 11 with advanced breast cancer) with MUC1 protein purified from the same patient from which the antibody was taken, from other patients or from healthy subjects. The healthy subjects used in this study were women who had no clinical and/or mammographical evidence of breast cancer. The reactivity of the monoclonal anti-MUC1 B55 antibody was measured as a comparative control. Antibodies produced by lymphocytes from four healthy subjects (N10 to N14) were used as a negative control.

The results presented demonstrate that B lymphocytes derived from patients with breast cancer produce autoantibodies that are able to recognise MUC1 protein isolated both from the same and from different patients. In addition, these autoantibodies bind with high specificity to MUC1 present in patients with cancer, showing almost no reactivity with MUC1 isolated from healthy individuals. These results are highly reproducible, since different autoantibodies show a very similar reactivity profile with MUC1 protein purified from different sources. Furthermore, the results obtained also indicate that the sensitivity of the autoantibodies for cancer-associated MUC1 is much greater than that observed for the monoclonal B55 antibody. Furthermore, antibodies produced by lymphocytes from normal patients did not show this profile.

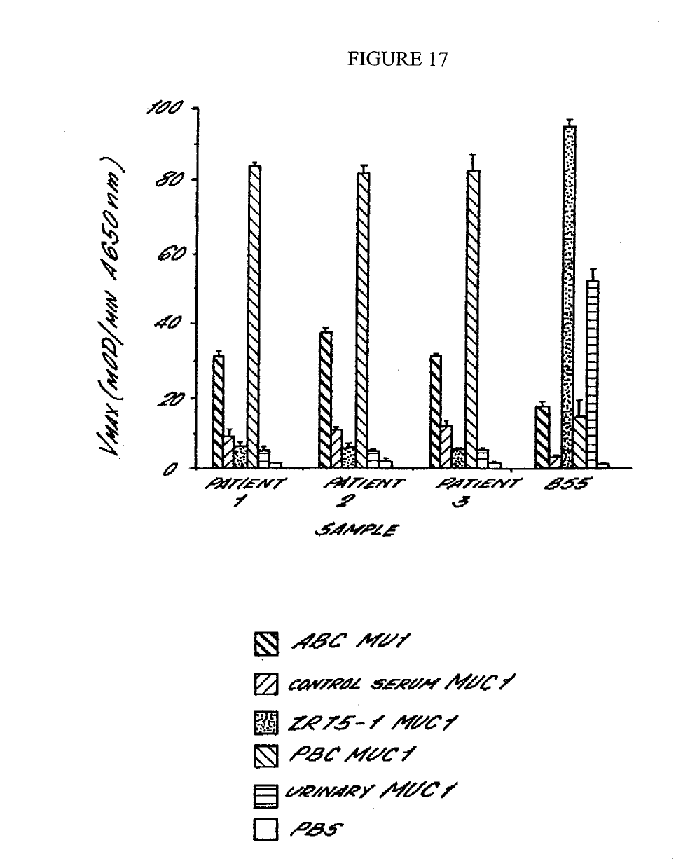

FIG. 17 shows the reactivity of autoantibodies secreted by immortalised B lymphocytes derived from patients with primary breast cancer with MUC1 protein from different sources, compared with that of B55. The reactivity of B55 is included as a comparative control. PBS is used as a negative control. The profile of reactivity of the different autoantibodies is again very reproducible. The autoantibodies show high specificity for MUC1 present in the serum of patients with cancer and have almost no affinity for MUC1 isolated from healthy individuals or from the breast cancer cell line ZR75-1. Furthermore, the affinity of the autoantibodies for MUC1 protein associated with either primary breast cancer or advanced breast cancer is much higher that measured for B55.

The data in FIG. 17 demonstrates the ability to obtain secreted human auto-antibodies from Epstein Barr Virus immortalized patient B cells.

### Example 19

### Measure of the Affinity of Autoantibodies with Surface Plasmon Resonance

Methods

Surface Plasmon Resonance was performed on Iasys Biosensor Plus (from Affinity Sensor). MUC1 protein from patients with advanced breast cancer and from normal individuals were adhered to amino silane coated cells following the manufacturers instructions and the cells were blocked with 1% (w/v) polyvinylpyrrolidone (PVP). Control cells coated only with 1% PVP were also produced. The binding of different dilutions of culture supernatant derived from Epstein Barr Virus transformed peripheral blood mononuclear B cells from patients with primary breast cancer was measured using the following experimental conditions:

| | | |
|---|---|---|
| Sampling interval: | 0.3 | msecs |
| Stirrer speed: | 70 | rpm |
| Temperature: | 24° | C. |
| Binding Time: | 3 | min |
| Dissociation with PBS: | 2 | minutes |
| Regeneration with 20 mM Hcl: | 3 | minutes |
| Re-equilibration with PBS: | 5 | minutes |

Results

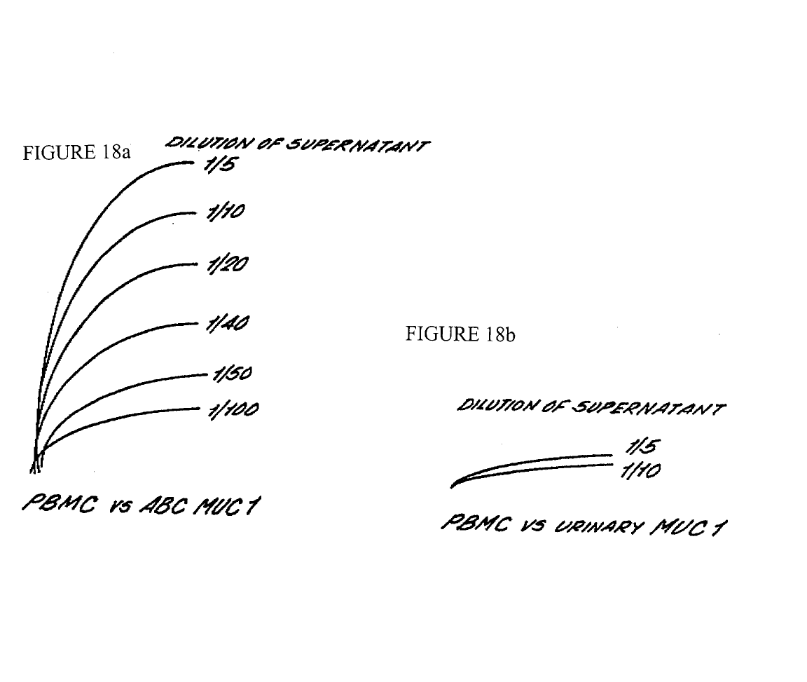

FIG. 18 shows that the autoantibodies produced by B lymphocytes derived from EBV transformed PBMC from a patient with primary breast cancer bind with a much higher affinity to MUC1 isolated from another patient with breast cancer than MUC1 isolated from a healthy individual.

### Example 20

### Detection of MUC1 Antigen in ELISA Assays Utilising Autoantibodies

Method:

1) Purification of Anti-MUC1 Autoantibodies from Sera

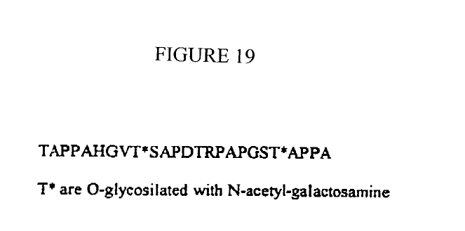

The MUC1 peptide TAP2, with the sequence shown in FIG. 19 was conjugated to CNBr-sepharose beads. Pooled sera from patients diagnosed with advanced breast cancer were diluted 1/10 in PBS and were incubated with the conjugated sepharose beads overnight at 4° C. with rolling (in the ratio of 25 ml of serum to 1 ml of beads). After centrifugation the supernatant was removed and the beads were washed 5 times with PBS or until absorbance at 280 nm was zero. Each wash was performed by resuspending the beads in PBS, rolling for 10 minutes, centrifuging and removing the supernatant. The beads were resuspended in 1 ml of 3M sodium thiocyanate in PBS, rolled at room temperature for 10 minutes and centrifuged. The supernatant was removed and dialysed against PBS at 4° C. The anti-MUC1 content was then confirmed by ELISA using as immobilised antigen both MCU1 isolated from patients with advanced breast cancer and a MUC1 peptide, with sequence APDTRTPAPG (SEQ ID NO:3) and conjugated to BSA.

2) Biotinylation of Anti-MUC1 Autoantibodies

The autoantibodies obtained as described above were concentrated to a volume of 100 μl by using centrifugal filters and then diluted to a volume of 1 ml with 0.1 sodium tetraborate buffer pH 8.8. 20 μg of N-hydroxysuccinimide biotin were added and the autoantibodies/biotin solution was incubated for 4 hours at room temperature with rolling. The reaction was stopped by addition of 10 μl of 1M NH₄Cl and incubation for ten minutes. The autoantibodies were then dialysed against PBS for thirty-six hours at 4° C. to remove unbound biotin. Aliquots of the autoantibodies solution were frozen and stored at −20° C. in the dark until use.

3) ELISA Assay

Culture supernatant of lymphocytes derived from patients with primary breast cancer or advanced breast cancer or the monoclonal anti-MUC1 C595 antibody were plated out at 50 μl per well in a 96 well microtitre assay plate and incubated overnight at 4° C. The plate was then washed 4 times with PBS/Tween, blocked for 60 minutes with a fresh solution of 2% (w/v) polyvinylpyrrolidone (PVP) in PBS and washed twice with PBS/Tween. 50 μl per well of MUC1 from different sources were added. After incubation at room temperature for sixty minutes, the plate was washed again four times with PBS/Tween. 50 μl of the appropriate biotinylated secondary antibody, either C595 or autoantibody purified from a pool of sera from a patient with advanced breast cancer, prepared as described above, were added to each well and incubated for 60 minutes at room temperature. After 4 washes with PBS/Tween, 50 μl of streptavidin-HRP were added to each well and incubated at room temperature for 60 minutes. The plate was again washed four times, 50 μl of TMB were added to each well and optical density (OD) at 650 nm for each well of the assay plate was read kinetically over a period of 10 minutes.

Results:

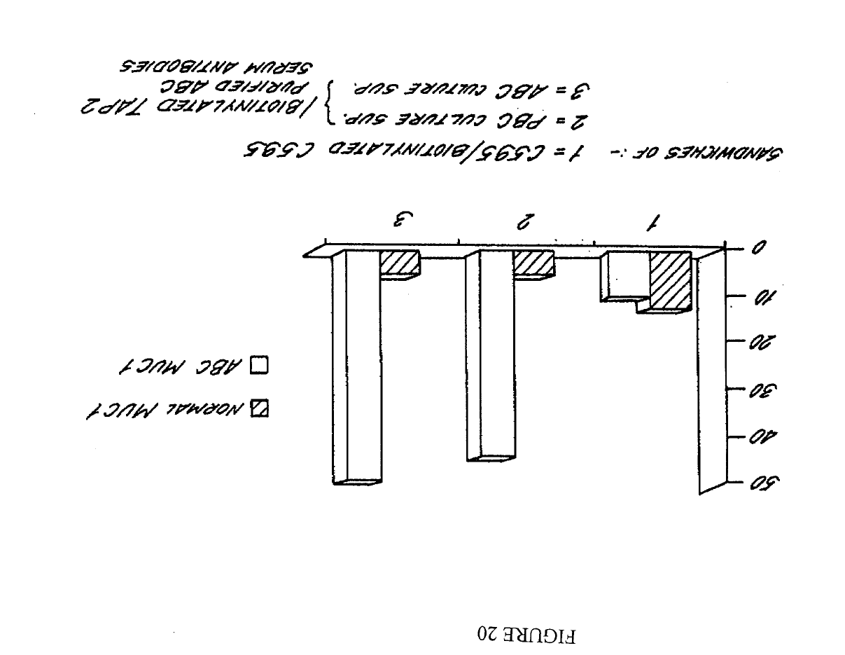

FIG. 20 shows the results of an ELISA assay utilising as immobilised antibodies autoantibodies produced by B lymphocytes derived from patients with primary or advanced breast cancer, compared with those obtained in a parallel assay with the monoclonal anti-MUC1 C595 antibody. The data indicate that autoantibodies from patients with breast cancer can be used in ELISA assays to specifically detect modified forms of MUC1 protein associated with cancer. These assays are more sensitive and show higher specificity than those utilising the monoclonal antibody C595.

### Example 21

### Use of the Assay to Detect MUC1 Proteins in Serum Samples of Patients

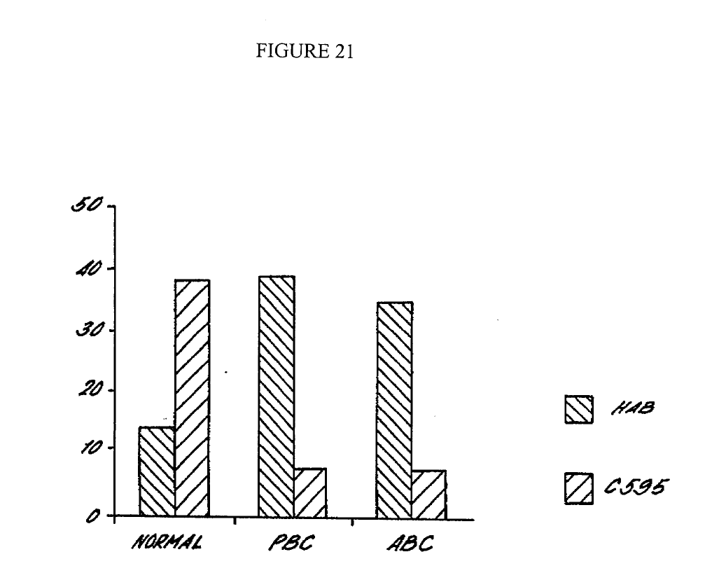

An ELISA assay was performed, as described in Example 20, on serum samples from healthy individuals or patients with primary or advanced breast cancer utilising as immobilised antibodies the autoantibodies produced by B lymphocytes derived from patients with primary breast cancer. A parallel assay utilising the monoclonal anti-MUC1 antibody C595 was performed on the same samples. The results, shown in FIG. 21, indicate that the assay employing autoantibodies is able to detect with high sensitivity MUC1 circulating in the blood of patients with breast cancer. In addition, contrary to utilising the monoclonal antibody C595, which was included as a comparative example, this assay has a very high specificity for cancer-associated forms of MUC1.

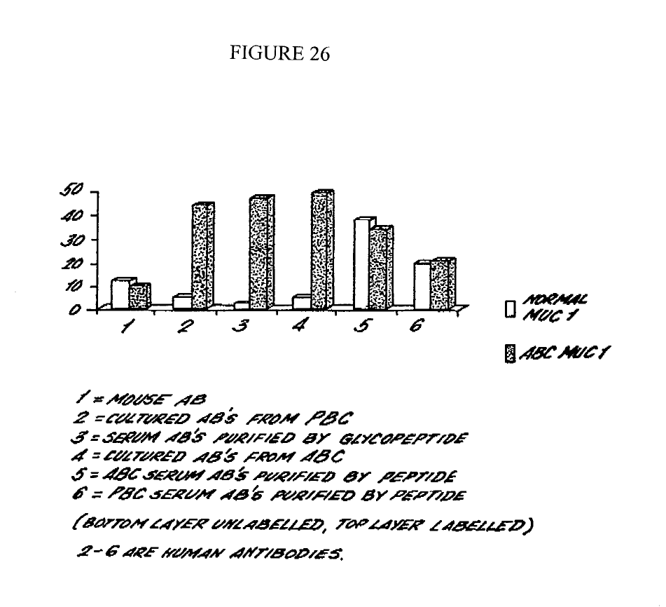

The data from examples 21 and 22 also shows that it is important to use MUC1 glycopeptide for purification of cancer-antigen specific antibodies, rather than a naked MUC1. The data in FIG. 26 also demonstrates the improved recognition of tumour associated MUC1 (ABC MUC1) seen using a sandwich ELISA utilising human auto-antibodies. Note that auto-antibodies immunoaffinity purified using a MUC1 glycopeptide retain their specificity whilst those immunoaffinity purified using a naked MUC1 peptide do not. Accordingly, another aspect of this invention is the provision of a method of purifying human auto-antibodies to human tumour marker proteins using the said tumour marker protein rather than a “normal” version. The method may be carried out using any of the tumour marker proteins mentioned herein.

### Example 22

### Use of the Assay to Monitor the Progression of the Disease

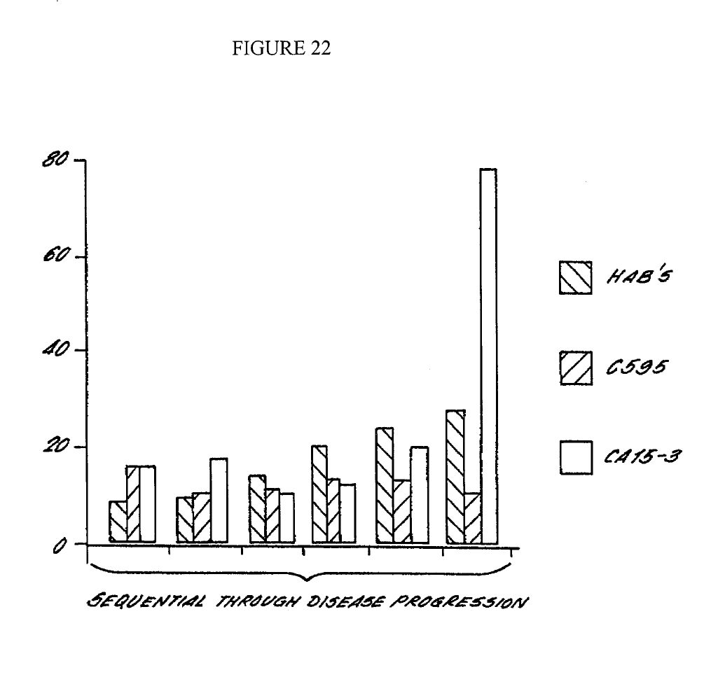

An ELISA assay was performed, as described in Example 18, on sequential serum samples from a patient diagnosed with metastatic cancer throughout the progression of the disease, using as immobilised antibodies the autoantibodies produced by B lymphocytes derived from patients with primary breast cancer or the monoclonal anti-MUC1 C595 antibody. Three different assay were used for the detection of circulating MUC1 in sequential serum samples taken from a single patient over the course of her disease (which was progressive in nature). The first two assays were designed with minimal optimisation purely to give an indication of the amount of detectable tumour associated MUC1, the first using human autoantibodies, the second using murine C595. The third assay used was a commercial assay using murine antibodies, considered to be the current ‘gold standard’, and performed by a Clinical Chemistry laboratory (the commercial CA15.3 assay). Three differing levels of detection were achieved with these assays and the results are shown in FIG. 22.

The laboratory developed human mouse assay detected low levels of MUC1 which increased over the time course, mimicking disease progression. Furthermore, rising levels of MUC1 were noted at the third time point.

The ‘gold standard’ commercial assay also indicated progressive disease by a dramatic increase in the level of MUC1, far greater than that detected by the human assay. However, rising levels were not detectable until the sixth sample, giving a reduced lead time over clinical detection compared to that give by the human assay. FIG. 22 shows that the assay employing autoantibodies can be used to follow the progression of cancer in a patient, wherein increasing levels of MUC1 detected in the assay indicate exacerbation of the disease. The data also demonstrate that the use of autoantibodies leads to results that better represent the development of the disease than those obtained with either the C595 antibody or the CA15-3 assay.

### Example 23

### Comparison of the Specificity of Anti-MUC1 Autoantibodies to Urinary or ABC MUC1

Method:

Preparations of ABC MUC1 (MUC1 isolated from the serum of patients diagnosed with advanced breast cancer) and urinary MUC1 were prepared as described in Example 18.

Aliquots of the ABC and urinary MUC1 preparations were dried onto the wells microtitre plates separately at concentrations giving equivalent NCRC-11 binding. After blocking with 2% PVP, serum samples taken from patients with breast cancer, diluted 1/100 with PBS, were added to the wells and any anti-MUC1 antibodies in the sera allowed to bind. After washing, the bound antibodies were probed with anti-human IgM-HRP and anti-human IgG-HRP conjugates.

Results

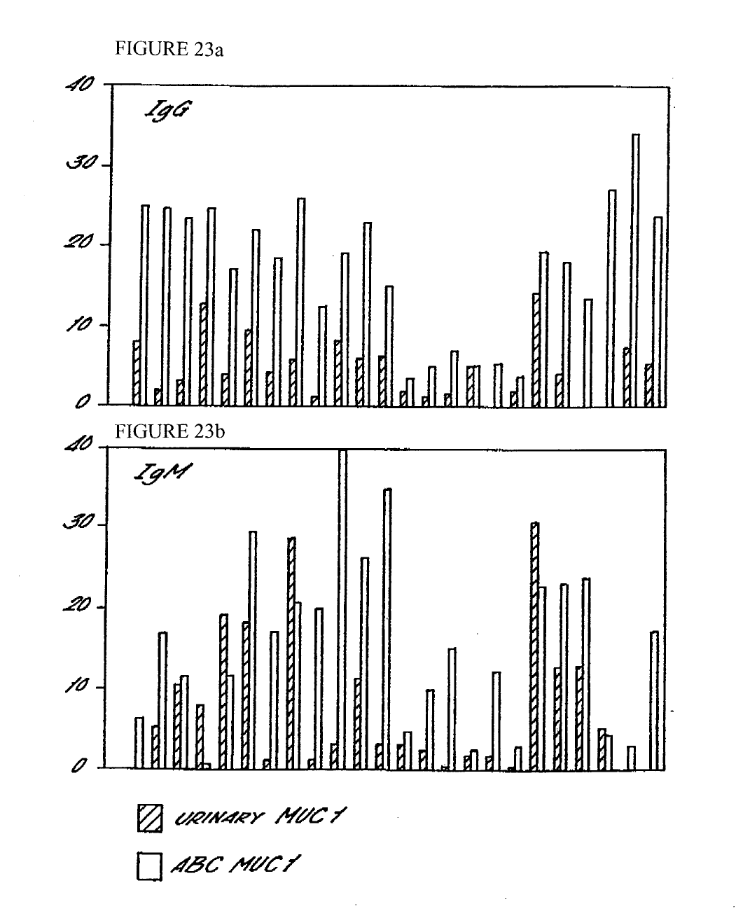

FIG. 23 shows the results of a number of determinations of reactivity of sera from breast cancer patients with ABC and urinary MUC1. Sera from the majority of patients clearly exhibit greater specificity for the ABC MUC1 as compared to urinary MUC1.

### Example 24

### IgG and IgM Responses

Specificity for Tumour Associated MUC1

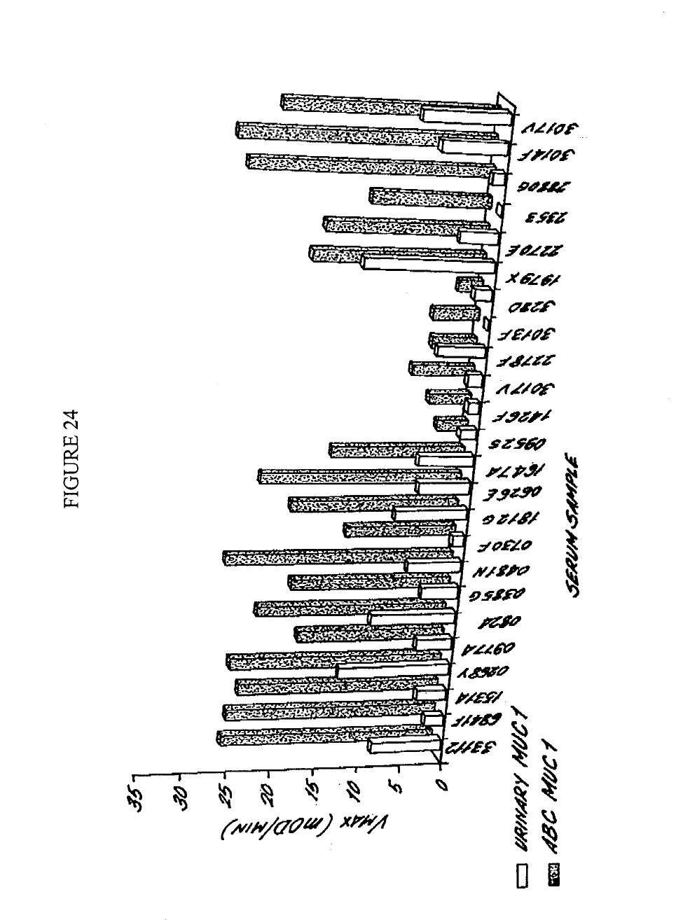

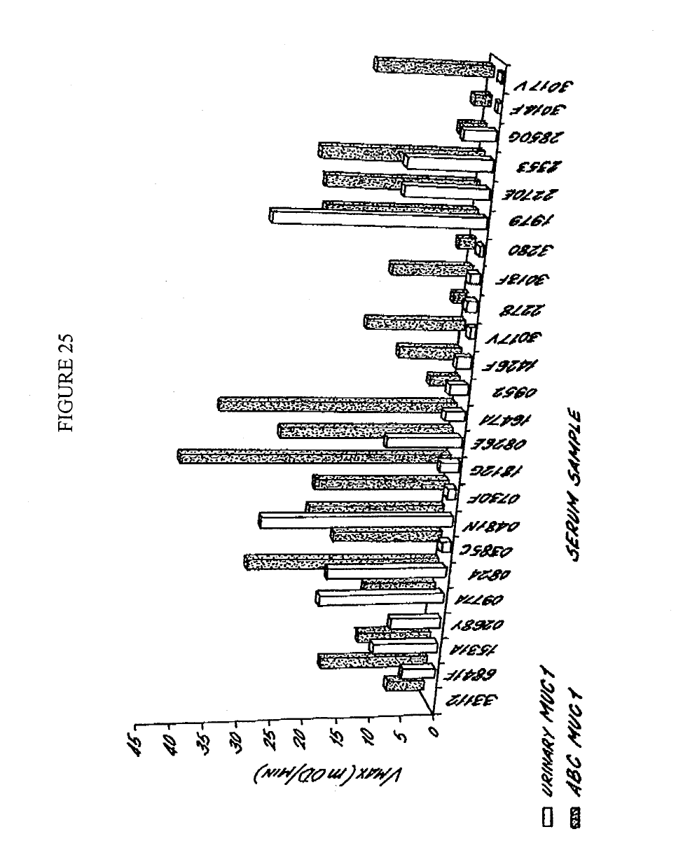

A cohort of patients with advanced breast cancer were assessed for their auto-antibody response to normal urinary MUC1 and tumour associated ABC MUC1. This assessment was sub-divided into IgG and IgM responses. For IgG response, 75% of patient demonstrated a significantly higher response towards tumour associated ABC MUC1 than to normal urinary MUC1. This held even in those patients whose anti-MUC1 response was weak. For IgM response, anti-tumour associated ABC MUC1 responses were higher in 66% of patients. The results are shown in FIGS. 24 and 25. These two figures provide evidence for the greater sensitivity of autoantibodies for tumour-associated MUC1 (ABC MUC1) as compared to normal urinary MUC1.

It should be understood that the foregoing disclosure emphasizes certain specific embodiments of the invention and that all modifications or alternatives equivalent thereto are within the spirit and scope of the invention set forth in the appended claims.

### Example 25

### Collection of Data

Blood and clinical data were collected from patients who attend an ‘At Risk’/Family History Clinics in the UK for patients perceived to be at increased risk of developing breast cancer, some of whom subsequently develop breast cancer are then treated at the Unit. Blood specimens and clinical data from patients attending the National Health Service Breast Screening Programme (NHSBSC) who are not known to be at any increased risk, other than that they are over 50 years of age was also collected.

Firstly, it was assessed whether there is an increase in expression of the markers using developed assays, in the ‘at risk’ group. Preliminary data was gathered to indicate whether expression is related to the:

a) Level of Risk

as determined by the strength of the family history,

as determined by any breast biopsy which shows significant pathological features indicating future cancer risk,

b) Development of Breast Cancer

as determined from the patients who develop breast cancer during this period. It was assessed whether the tumours which have developed express any of the markers measured in the serum. From the data accumulated thus far there is expected to be a positive correlation between tumour tissue expression and detection in the serum of a marker or auto-antibody.

Assays for BRCA1 and 2 auto-antibody detection are being run in this population of women to confirm that they should be added to the panel, especially in those women with a very strong family history suggestive of the presence of a gene mutation.

Secondly, if the serum measurements correlate significantly with the level of risk and/or development of breast cancer as described above, analysis of the data collected on the normal population attending the National Health Service Breast Screening Programme will be carried out. This would assess if the same serum measurements in this latter group also selected out patients who would be at ‘increased risk’ or who would definitely develop breast cancer.

Thirdly, whether sequential serum measurements show any correlation with the onset of the process of carcinogenesis and the development of breast cancer was formally determined.

By combining the results of auto-antibody detection against different cancer-associated markers, both the sensitivity and specificity for breast cancer is improved.

### REFERENCES

1. Early Breast Cancer Trialists' Cooperative Group Systemic treatment of early breast cancer by hormonal, cytotoxic and immune therapy. The Lancet 1992; 1: 1-15

2. Early Breast Cancer Trialists' Cooperative Group The Lancet 1992; 1:71-85

3. Jager W, Kramer S, Lang N. Disseminated breast cancer: does early treatment prolong survival without symptoms. The Breast 1995; 4:65

4. Molina, R. Personal Communication at Tumour Marker Workshop, September 1995

5. Nicolini A, Anselmi L, Michelassi C, Carpi A. Prolonged survival by early salvage treatment of breast cancer patients: a retrospective 6 year study. Br. J. Cancer 1997; 76:1106-1111

6. Roberston J F R. Personnel communication.

7. Robertson J F R, Person D, Price M R, Selby C, Blarney R W, Howell A. Objective measurement of therapeutic response in breast cancer using serum markers. Br J Cancer 1991; 64:757-763

8. Robertson J F R, Pearson D, Price M R, Selby C, Pearson J, Blarney R W, Howell A. Prospective assessment of the role of five serum markers in breast cancer. Cancer Immunol & Immunotherapy 1991b; 33:403-410

9. Robertson J F R. Role of serum tumour markers in breast cancer. The Breast 1995; 4:62-63

10. Robertson J F R, Jaeger W, Syzmendera J J, Selby C, Coleman R, Howell A, Winstanley J, Jonssen P E, Bombardieri E, Sainsbury J R C, Gronberg H, Kumpulainen E, Blarney R W. The objective measurement of remission and progression in metastatic breast cancer by the use of serum tumour markers. Eur. J. Cancer 1999; 35:47-53

11. Dixon AR, Jonrup I, Jackson L, Chan S Y, Badley R A, Blarney R W. Serological monitoring of advanced breast cancer treated by systemic cytotoxics by a combination of CEA, CA15.3 and ESR:fact or fiction? Disease Markers 1991; 9:167-174

12. Dixon A R. MD Thesis, Nottingham University; May 1992

13. Dixon A R, Jackson L, Chan S Y, Badley R A, Blarney R W. Continuous chemotherapy in responsive metastatic breast cancer: a role for tumour markers? Br. J. Cancer 1993; 68:121-129

14. Kotera Y, Fontenot J D, Precher G, Metzgar R S, Finn O J. Humoral immunity against a tandem repeat epitope of human mucin MUC1 in sera from breast, pancreatic and colon cancer patients. Cancer Reasearch 1994; 54:2856-2860

15. Gourevitch M Mj, von Mensdorff-Pouilly S, Livinov S V, Kenemans P, van Kamp G J, Verstraeten A A, Hilgers J. Polymorphic epithelial mucin (MUC1) containing circulating immune complexes in carcinoma patients. Br. J. Cancer 1995; 72:943-938

16. Croce M V, Price M R, Segal-Eiras A. Expression of monoclonal antibody defined-antigens in fractions isolated from human breast carcinomas and patients serum. Cancer Immunol & Immunotherapy 1995; 40:132-13

17. Chen X Q, Stroun M, Magnenat J L, Nicod L P, Kurt A M, Lyautey J, Lederry C, Anker P. Microsatellite alterations in plasma DNA of small cell lung cancer patients. Nature Medicine 1996; 2:1033-1034

18. Nawroz H, Koch W, Anker P, Stroun M, Sidransky D. Microsatellite alterations in serum DNA of head and neck of cancer patients. Nature Medicine 1996; 2:1033-1034

19. Robertson J F R, Whynes D K, Dixon A, Blamey R W. Potential for cost economies in guiding therapy in patients with metastatic breast cancer. Br. J. Cancer 1995; 72:174-177

1. A method of detecting the immune response of a mammal to circulating tumour marker proteins or tumour cells expressing said tumour marker proteins, which method comprises steps of:
(a) contacting a sample of bodily fluids from said mammal with one or more distinct tumour marker antigens selected from the group consisting of PTH-RP, CYFRA 21-1, kallikrein, pro-gastrin, gastrin G17, gastrin G34, CA19-9, CA72-4, vasopressin, gastrin releasing peptide, SSC, TK, αFP, p62, annexins I and II, Hu, KOC, an antigen of HPV and any protein or polypeptide expressed by one of the CEA gene family members or an epitopic fragment of any of the above;
(b) determining the presence or absence of complexes of said tumour marker antigen or antigens bound to autoantibodies present in said sample of bodily fluids, said autoantibodies being immunologically specific to said tumour marker proteins;
whereby the presence of said complexes is indicative of the immune response to circulating tumour marker proteins or tumour cells expressing said tumour marker proteins.

2. A method as claimed in claim 1 wherein said tumour marker antigen is labelled with a protein or peptide tag.

3. A method as claimed in claim 1 or claim 2 wherein said tumour marker antigen is labelled with biotin.

4. A method as claimed in claim 1 wherein at least one of said tumour marker antigens is used in combination with other tumour marker antigens selected from the group consisting of MUC1, p53, c-erbB2, Ras, c-myc, BRCA1, BRCA2, PSA, APC and CA125.

5. A method as claimed in claim 4 wherein the autoantibodies detected are indicative of cancer, preferably breast, bladder, colorectal, prostate, ovarian, lung, liver, pancreatic, gastric, oesophageal or cervical cancer.

6. An assay kit suitable for performing the assay method as claimed in claim 1, said assay kit comprising one or of more distinct tumour marker antigens selected from the group consisting of PTH-RP, CYFRA 21-1, kallikrein, pro-gastrin, gastrin G17, gastrin G34, CA19-9, CA72-4, vasopressin, gastrin releasing peptide, SSC, TK, αFP, p62, annexins I and II, Hu, KOC, an antigen of HPV and any protein or polypeptide expressed by one of the CEA gene family members or an epitopic fragment of any of the above and means for contacting said one or more antigens with a sample of bodily fluids.

7. Use of the method of claim 1 in the detection of cancer, preferably breast, bladder, colorectal, prostate, ovarian, liver, lung, pancreatic, gastric, oesophageal or cervical cancer.

8. Use of the method of claim 1 in the detection of early neoplastic or early carcinogenic change in asymptomatic patients in the detection of recurrent disease in a patient previously diagnosed as carrying tumour cells, which patient has undergone treatment to reduce the number of said tumour cells, in the identification of those individuals which are at increased risk of developing cancer in a population of asymptomatic individuals, in determination of the tumour marker profile of an individual suffering from cancer, in predicting the response of an individual with cancer to an anti-cancer treatment or in selecting the most suitable therapy for an individual with cancer.

9. The use as claimed in claim 8 wherein tumour marker profile of an individual suffering from cancer, or at risk of suffering from cancer is determined sequentially in said individual as an indication of the course of disease.

10. The use as claimed in claim 8 wherein said anti-cancer treatment is hormone therapy, chemotherapy, radiotherapy, anti-growth factor therapy, immune therapy or vaccination.

11. A method of determining the immune response of a patient to two or more circulating tumour marker proteins or to tumour cells expressing said tumour marker proteins and identifying which one or more of said two or more tumour marker proteins elicits the strongest immune response in said patient, which method comprises steps of:
(a) contacting a sample of bodily fluids from said patient with a panel of two or more distinct tumour marker antigens; selected from the group consisting of PTH-RP, CYFRA 21-1, kallikrein, pro-gastrin, gastrin G 17, gastrin G34, CA19-9, CA72-4, vasopressin, gastrin releasing peptide, SSC, TK, αFP, p62, annexins I and II, Hu, KOC, an antigen of HPV and any protein or polypeptide expressed by one of the CEA gene family members or an epitopic fragment of any of the above;
(b) measuring the amount of complexes formed by binding of each of said tumour marker antigens to autoantibodies present in said sample of bodily fluids, said autoantibodies being immunologically specific to said tumour marker proteins;
(c) using the measurement obtained in part (b) as an indicator of the relative strength of the immune response to each tumour marker protein and thereby identifying which one of said two or more tumour marker proteins elicits the strongest immune response in said patient.

12. A method as claimed in claim 11 wherein at least one of said tumour marker antigens is labelled with a protein or peptide tag.

13. A method as claimed in claim 11 wherein at least one of said tumour marker antigens is labelled with biotin.

14. A method as claimed in claimed 11 wherein said two or more tumour marker antigens are selected from the group consisting of MUC1, c-erbB2, c-myc, Ras, p53, BRCA1, BRCA2, PSA, APC and CA125.

15. Use of the method of claim 11 in the selection of a course of vaccine treatment.

16. Use as claimed in claim 11 wherein one or more tumour marker proteins identified as eliciting a strong immune response in said patient is/are used as the basis of a vaccine treatment.

17. A method of determining whether a vaccination procedure comprising challenging a patient with an immunogenic preparation comprising an antigenic tumour associated-protein selected from the group consisting of PTH-RP, CYFRA 21-1, kallikrein, pro-gastrin, gastrin G17, gastrin G34, CA19-9, CA72-4, vasopressin, gastrin releasing peptide, SSC, TK, αFP, p62, annexins I and II, Hu, KOC, an antigen of HPV and any protein or polypeptide expressed by one of the CEA gene family members or an epitopic fragment of any of the above or an antigenic fragment thereof has been successful in eliciting a humoral immune response, which method comprises the steps of:
(a) contacting a sample of serum from said patient with the said tumour associated protein;
(b) determining the presence or absence of complexes of said tumour associated protein bound to antibodies present in said sample of serum, said antibodies being immunologically specific to said tumour associated protein or an antigenic fragment thereof;
whereby the presence of said complexes indicates that said vaccination procedure has been successful in eliciting a humoral immune response.

18. An in vitro method for detecting a cancer-associated marker protein present in a bodily fluid of a mammal, said cancer-associated marker protein being selected from the group consisting of an antigenic tumour associated-protein selected from the group consisting of PTH-RP, CYFRA 21-1, kallikrein, pro-gastrin, gastrin G17, gastrin G34, CA19-9, CA72-4, vasopressin, gastrin releasing peptide, SSC, TK, αFP, p62, annexins I and II, Hu, KOC, an antigen of HPV and any protein or polypeptide expressed by one of the CEA gene family members or an epitopic fragment of any of the above which method comprises the steps of:
(a) contacting a sample of bodily fluid from said mammal with antibodies directed against at least one epitope of said marker protein; and
(b) detecting the presence of any complexes formed between said antibodies and any marker protein present in said sample;
wherein said antibodies are mammalian autoantibodies to said cancer-associated marker protein which are derived from the same species as the mammal from which said sample has been obtained.

19. A method as claimed in claim 18 wherein said sample is from a mammal substantially asymptomatic for pre-neoplasia or cancer, symptomatic for cancer, or from a mammal which has received therapy for cancer.

20. A method as claimed in claim 18 wherein the mammal is a human and the autoantibodies are human autoantibodies.

21. A method as claimed in claim 18 wherein said bodily fluid is plasma, serum, whole blood, urine, faeces, lymph, cerebrospinal fluid or nipple aspirate.

22. A method as claimed in claim 18 wherein said cancer-associated marker protein is associated with lymphomas, leukaemias, breast cancers, colorectal cancers, liver cancers, lung cancers, pancreatic cancers, prostate cancers, cervical cancers, ovarian cancers, endometrial cancers or cancers of the skin.

23. A method as claimed in claim 18 wherein said cancer-associated marker protein is a breast cancer-associated marker protein.

24. A method as claimed in claim 18 wherein said autoantibodies are obtainable from monocytes isolated from a patient with primary breast cancer.

25. A method as claimed in claim 18 wherein said autoantibodies are obtainable from monocytes isolated from a patient with advanced breast cancer.

26. A method as claimed in claim 18 wherein said autoantibodies are produced by an immortalized cell or cell population.

27. A method as claimed in claim 18 wherein said autoantibodies are polyclonal antibodies.

28. A method as claimed in claim 18 wherein said autoantibodies are immobilized on a solid surface.

29. A method as claimed in claim 28 wherein any complexes formed between said autoantibodies and any cancer-associated marker protein present in said sample are detected using secondary antibodies or autoantibodies specific for at least one epitope of said marker protein, said secondary autoantibodies carrying a detectable label.

30. A method as claimed in claim 28 wherein in addition to said sample a labelled cancer-associated marker protein is added carrying at least one epitope recognised by said autoantibodies.

31. Use of a method as claimed in claim 18 to screen for recurrence of cancer after a treatment, to monitor systemic therapies or to select therapies.

32. A diagnostic reagent which comprises mammalian autoantibodies with a specificity for at least one epitope of a mammalian cancer-associated marker protein selected from the group consisting of PTH-RP, CYFRA 21-1, kallikrein, pro-gastrin, gastrin G17, gastrin G34, CA19-9, CA72-4, vasopressin, gastrin releasing peptide, SSC, TK, αFP, p62, annexins I and II, Hu, KOC, an antigen of HPV and any protein or polypeptide expressed by one of the CEA gene family members or an epitopic fragment of any of the above.

33. A reagent as claimed in claim 32 wherein said autoantibodies are human autoantibodies and said marker protein is a human cancer-associated marker protein.

34. A reagent as claimed in claim 32 wherein said autoantibodies have specificity for at least one epitope of a cancer-associated marker protein associated with lymphomas, leukaemias, breast cancers, colorectal cancers, lung cancers, liver cancers, pancreatic cancers, prostate cancers, cervical cancers, ovarian cancers, endometrial cancers or cancers of the skin.

35. A reagent as claimed in claim 32 wherein said autoantibodies have specificity for at least one epitope of a breast cancer-associated marker protein.

36. A reagent as claimed in claim 32 wherein said autoantibodies are obtainable from monocytes isolated from a patient with primary breast cancer.

37. A reagent as claimed in claim 32 wherein said autoantibodies are obtainable from monocytes isolated from a patient with advanced breast cancer.

38. An immortalized cell population capable of producing autoantibodies directed against at least one epitope of a mammalian cancer-associated marker protein selected from the group consisting of an antigenic tumour associated-protein selected from the group consisting of PTH-RP, CYFRA 21-1, kallikrein, pro-gastrin, gastrin G17, gastrin G34, CA19-9, CA72-4, vasopressin, gastrin releasing peptide, SSC, TK, αFP, p62, annexins I and II, Hu, KOC, an antigen of HPV and any protein or polypeptide expressed by one of the CEA gene family members or an epitopic fragment of any of the above.

39. An immortalized cell population as claimed in claim 38 which is capable of producing autoantibodies directed against at least one epitope of a human cancer-associated marker protein.

40. An immortalized cell population as claimed in claim 38 wherein said autoantibodies are directed against at least one epitope of a cancer-associated marker protein associated with lymphomas, leukaemias, breast cancers, colorectal cancers, lung cancers, liver cancers, pancreatic cancers, prostate cancers, cervical cancers, ovarian cancers, endometrial cancers or cancers of the skin.

41. An immortalised cell population as claimed in claim 38 wherein said autoantibodies are directed against an epitope of a breast cancer-associated marker protein.

42. An immortalized cell population as claimed in claim 38 which is derived from monocytes isolated from a patient or a group of patients having cancer or other neoplasia.

43. An immortalised cell population as claimed in claim 38 wherein said cell population is derived from monocytes of a patient or group of patients having primary breast cancer.

44. An immortalised cell population as claimed in claim 38 wherein said cell population is derived from monocytes of a patient or group of patients with advanced breast cancer.

45. A kit for detecting a cancer-associated marker protein present in a bodily fluid of a mammal, the kit comprising:
(a) mammalian autoantibodies directed against a cancer-associated marker protein from the same species as said autoantibodies said cancer-associated marker protein being selected from the group consisting of an antigenic tumour associated-protein selected from the group consisting of PTH-RP, CYFRA 21-1, kallikrein, pro-gastrin, gastrin G17, gastrin G34, CA19-9, CA72-4, vasopressin, gastrin releasing peptide, SSC, TK, αFP, p62, annexins I and II, Hu, KOC, an antigen of HPV and any protein or polypeptide expressed by one of the CEA gene family members or an epitopic fragment of any of the above; and
(b) means for detecting the formation of complexes between said autoantibodies and said cancer-associated marker protein.

46. A kit as claimed in claim 45 wherein said autoantibodies are human autoantibodies

47. A kit as claimed in claim 45 wherein said marker protein is a cancer-associated marker protein associated with lymphomas, leukaemias, breast cancers, colorectal cancers, lung cancers, liver cancers, pancreatic cancers, prostate cancers, cervical cancers, ovarian cancers, endometrial cancers or cancers of the skin.

48. A kit as claimed in claim 45 wherein said marker protein is a breast-cancer associated marker protein.

49. A method of treating or of preventing cancer which method comprises administering to a mammal an effective amount of an auto-antibody to a cancer-associated antigen.

50. A method as claimed in claim 49 wherein said mammal is human.

51. A method as claimed in claim 49 wherein said auto-antibody is a human auto-antibody.

52. A method as claimed in claim 49 wherein said cancer is selected from a group consisting of lymphomas, leukaemias, breast cancer, colorectal cancers, lung cancers, liver cancers, pancreatic cancers, prostate cancers, cervical cancers, ovarian cancers, endometrial cancers or cancers of the skin.

53. A method as claimed in claim 49 wherein said cancer-associated antigen is selected from the group consisting of MUC1, c-erbB2, c-myc, p53, ras, BRCA1, BRCA2, APC, CA125, PSA, PTH-RP, CYFRA21-1, kallikrein, pro-gastrin, gastrin G17, gastrin F34, CA19-9, CA72-4, vasopressin, gastrin releasing peptide, SCC, TK, αFP, p62, annexins I and II, Hu, KOC, an antigen of HPV and any protein or polypeptide expressed by one of the CEA gene family.

54. A method as claimed in claim 49 wherein said auto-antibody is obtainable from monocytes isolated from a patient suffering with cancer.

55. A method as claimed in claim 49 wherein said auto-antibody is produced by an immortalized cell or cell population.

56. A method as claimed in claim 49 wherein said auto-antibody has attached thereto, reversibly or otherwise, a therapeutic agent (e.g. a cytotoxic agent).

57. A pharmaceutical composition comprising an auto-antibody to cancer-associated antigen and a pharmaceutically acceptable carrier or diluent.

58. A composition as claimed in claim 57 wherein said auto-antibody is a human auto-antibody.

59. A composition as claimed in claim 57 which is for use in treatment or prophylaxis of a cancer selected from the group consisting of lymphomas, leukaemias, breast cancer, colorectal cancers, lung cancers, pancreatic cancers, prostate cancers, cervical cancers, ovarian cancers, endometrial cancers or cancers of the skin.

60. A composition as claimed in claim 57 wherein said cancer-associated antigen is selected from the group consisting of MUC1, c-erbB2, c-myc, p53, ras, BRCA1, BRCA2, APC, CA125, PSA, PTH-RP, CYFRA21-1, kallikrein, pro-gastrin, gastrin G17, gastrin F34, CA19-9, CA72-4, vasopressin, gastrin releasing peptide, SCC, TK, αFP, p62, annexins I and II, Hu, KOC, an antigen of HPV and any protein or polypeptide expressed by one of the CEA gene family.

61. A composition as claimed in claim 57 wherein said auto-antibody is obtainable from monocytes isolated from a patient with cancer or is produced by an immortalized cell or cell population.

62. A composition as claimed in claim 57 wherein said auto-antibody has attached thereto, reversibly or otherwise, a therapeutic agent e.g. cytotoxic agent.

63. A method of diagnosing cancer which method comprises administering to a mammal, a diagnostic reagent which comprises an auto-antibody to a cancer-associated antigen, said antibody having attached thereto an imaging agent.

64. A method as claimed in claim 63 wherein said mammal is a human.

65. A method as claimed in claim 64 wherein said auto-antibody is a human auto-antibody.

66. A method as claimed in claim 65 wherein said cancer is selected from the group consisting of lymphomas, leukaemias, breast cancer, colorectal cancers, lung cancers, liver cancers, pancreatic cancers, prostate cancers, cervical cancers, ovarian cancers, endometrial cancers or cancers of the skin.

67. A method as claimed in claim 63 wherein said cancer-associated antigen is selected from the group consisting of MUC1, c-erbB2, c-myc, p53, ras, BRCA1, BRCA2, APC, CA125, PSA, PTH-RP, CYFRA21-1, kallikrein, pro-gastrin, gastrin G17, gastrin F34, CA19-9, CA72-4, vasopressin, gastrin releasing peptide, SCC, TK, αFP, p62, annexins I and II, Hu, KOC, an antigen of HPV and any protein or polypeptide expressed by one of the CEA gene family.

68. A method as claimed in claim 63 wherein said auto-antibody is obtainable from monocytes isolated from a patient with cancer or is produced by an immortalized cell or cell population.

69. A method of vaccinating a mammal against cancer which comprises administering said individual an effective amount of a cancer-associated antigen comprising at least one cancer-associated epitope, defined by an autoantibody.

70. A method of claim 69 wherein a epitopic fragment of a cancer-associated antigen is administered to a mammal.

71. A method as claimed in claim 69 wherein said cancer is selected from the group consisting of lymphomas, leukaemias, breast cancer, colorectal cancers, lung cancers, liver cancers, pancreatic cancers, prostate cancers, cervical cancers, ovarian cancers, endometrial cancers or cancers of the skin.

72. A method as claimed in claim 69 wherein said cancer-associated antigen is selected from the group consisting of MUC1, c-erbB2, c-myc, p53, ras, BRCA1, BRCA2, APC, CA125, PSA, PTH-RP, CYFRA21-1, kallikrein, pro-gastrin, gastrin G17, gastrin F34, CA19-9, CA72-4, vasopressin, gastrin releasing peptide, SCC, TK, αFP, p62, annexins I and II, Hu, KOC, an antigen of HPV and any protein or polypeptide expressed by one of the CEA gene family.

73. A method as claimed in claim 72 wherein an epitopic fragment of said cancer-associated antigesn is administered.

74. A method as claimed in claim 69 wherein said mammal is human.

75. An anti-cancer vaccine comprising a cancer-associated antigen or an epitopic fragment thereof, defined by an autoantibody and a pharmaceutically acceptable carrier or diluent.

76. A vaccine as claimed in claim 75 wherein said cancer is selected from the group consisting of lymphomas, leukaemias, breast cancer, colorectal cancers, lung cancers, liver cancers, pancreatic cancers, prostate cancers, cervical cancers, ovarian cancers, endometrial cancers or cancers of the skin.

77. A vaccine as claimed in claim 75 wherein said cancer-associated-antigen is selected from the group consisting of MUC1, c-erbB2, c-myc, p53, ras, BRCA1, BRCA2, APC, CA125, PSA, PTH-RP, CYFRA21-1, kallikrein, pro-gastrin, gastrin G17, gastrin F34, CA19-9, CA72-4, vasopressin, gastrin releasing peptide, SCC, TK, αFP, p62, annexins I and II, Hu, KOC, an antigen of HPV and any protein or polypeptide expressed by one of the CEA gene family.

78. A method of purifying mammalian auto-antibodies to mammalian cancer-associated antigens which comprises using the said cancer-associated antigen or an epitopic fragment thereof as an immunoaffinity reagent.

79. A method as claimed in claim 78 wherein said cancer-associated antigen is selected from the group consisting of MUC1, c-erbB2, c-myc, p53, ras, BRCA1, BRCA2, APC, CA125, PSA, PTH-RP, CYFRA21-1, kallikrein, pro-gastrin, gastrin G17, gastrin F34, CA19-9, CA72-4, vasopressin, gastrin releasing peptide, SCC, TK, αFP, p62, annexins I and II, Hu, KOC, an antigen of HPV and any protein or polypeptide expressed by one of the CEA gene family.
